(19) United States
(12) Patent Application Publication
(10) Pub. No.: US 2009/0136827 A1
(43) Pub. Date: May 28, 2009

(54) ENZYMES IMMOBILIZED IN HYDROPHOBICALLY MODIFIED POLYSACCHARIDES

(75) Inventors: Shelley D. Minteer, Pacific, MO (US); Tamara L. Klotzbach, Lake Village, IN (US); Rodica Duma, St. Louis, MO (US)

Correspondence Address: SENNIGER POWERS LLP, 100 NORTH BROADWAY, 17TH FLOOR, ST LOUIS, MO, 63102, US

(73) Assignee: ST. LOUIS UNIVERSITY, St. Louis, MO (US)

(21) Appl. No.: 12/092,115

(22) PCT Filed: Nov. 2, 2006
(86) PCT No.: PCT/US2006/060487

§ 371 Date: Sep. 30, 2008

Related U.S. Application Data
(60) Provisional application No. 60/732,473, filed on Nov. 2, 2005.

(51) Int. Cl. H01M 4/90; C12N 11/10; C07H 1/00

(52) U.S. Cl. 429/43; 435/178; 536/123.1

(57) ABSTRACT

Bioanodes, biocathodes, biofuel cells, immobilized enzymes and immobilization materials comprising a micellar hydrophobically modified polysaccharide are disclosed. In particular, the micellar hydrophobically modified polysaccharide can be a hydrophobically modified chitosan or a hydrophobically modified alginate.

This invention was made with Government support under Grant No. 3-00475 awarded by the Office of Navel Research, Grant No. 3-00487 awarded by the Defense Advanced Research Projects Agency, and Grant No. 300477 awarded by the U.S. Central Intelligence Agency. The Government has certain rights in the invention.

### BACKGROUND OF THE INVENTION

The present invention is directed in general to biological enzyme-based fuel cells (a.k.a. biofuel cells) and their methods of manufacture and use. More specifically, the invention is directed to bioanodes, biocathodes, biofuel cells, immobilized enzymes, and enzyme immobilization materials comprising hydrophobically modified polysaccharides and their method of manufacture and use.

A biofuel cell is an electrochemical device in which energy derived from chemical reactions is converted to electrical energy by means of the catalytic activity of living cells and/or their enzymes. Biofuel cells generally use complex molecules to generate at the anode the hydrogen ions required to reduce oxygen to water, while generating free electrons for use in electrical applications. A bioanode is the electrode of the biofuel cell where electrons are released upon the oxidation of a fuel and a biocathode is the electrode where electrons and protons from the anode are used by the catalyst to reduce peroxide or oxygen to water. Biofuel cells differ from the traditional fuel cell by the material used to catalyze the electrochemical reaction. Rather than using precious metals as catalysts, biofuel cells rely on biological molecules such as enzymes to carry out the reaction.

### SUMMARY OF THE INVENTION

Among the various aspects of the present invention is a bioanode comprising an electron conductor; at least one anode enzyme; and an enzyme immobilization material. The anode enzyme is capable of reacting with an oxidized form of an electron mediator and the fuel fluid to produce an oxidized form of the fuel fluid and a reduced form of the electron mediator. The reduced form of the electron mediator is capable of releasing electrons to the electron conductor. The enzyme immobilization material is permeable to the fuel fluid and the electron mediator and comprises a hydrophobically modified polysaccharide.

In another aspect of the anode described above, the enzyme immobilization material comprises the electron mediator.

Yet another aspect is a bioanode comprising an electron conductor; at least one anode enzyme; an enzyme immobilization material; and an electrocatalyst. The anode enzyme is capable of reacting with an oxidized form of an electron mediator and the fuel fluid to produce an oxidized form of the fuel fluid and a reduced form of the electron mediator. The enzyme immobilization material is permeable to the fuel fluid and the electron mediator, and comprises a hydrophobically modified polysaccharide. The electrocatalyst is adjacent the electron conductor. An oxidized form of the electrocatalyst is capable of reacting with the reduced form of the electron mediator to produce an oxidized form of the electron mediator and a reduced form of the electrocatalyst, and the reduced form of the electrocatalyst is capable of releasing electrons to the electron conductor.

In another aspect of the anode described above, the enzyme immobilization material comprises the electron mediator, the electrocatalyst, or the electron mediator and the electrocatalyst.

A further aspect is a biocathode comprising an electron conductor; at least one cathode enzyme; and an enzyme immobilization material. The cathode enzyme is capable of reacting with a reduced form of an electron mediator and an oxidant to produce an oxidized form of the electron mediator and water. The enzyme immobilization material comprises the electron mediator, is permeable to the oxidant, and comprises a hydrophobically modified polysaccharide. An oxidized form of the electron mediator is capable of gaining electrons from the electron conductor to produce a reduced form of the electron mediator.

Yet another aspect of the invention is a biocathode comprising an electron conductor; at least one cathode enzyme; and an enzyme immobilization material. The cathode enzyme is capable of reacting with a reduced form of an electron mediator and an oxidant to produce an oxidized form of the electron mediator and water. The enzyme immobilization material comprises an electron mediator, an electrocatalyst, or an electron mediator and an electrocatalyst, is permeable to the oxidant, and comprises a hydrophobically modified polysaccharide. An oxidized form of the electrocatalyst being capable of gaining electrons from the electron conductor to produce a reduced form of the electrocatalyst that is capable of reacting with an oxidized form of the electron mediator to produce a reduced form of the electron mediator and an oxidized form of the electrocatalyst.

A further aspect is a biofuel cell for generating electricity comprising a fuel fluid; an electron mediator; a bioanode as described above; and a cathode. Further, a biofuel cell for generating electricity comprising a fuel fluid; an electron mediator; an anode; and a biocathode as described above. Also, a biofuel cell for generating electricity comprising a fuel fluid; an electron mediator; a bioanode as described above; and a biocathode as described above.

Yet another aspect is a method of generating electricity using the biofuel cells described above comprising oxidizing the fuel fluid at the anode or bioanode and reducing the oxidant at the cathode or biocathode; oxidizing the reduced form of the electron mediator during the reduction of the oxidant at the cathode or biocathode; oxidizing the electrocatalyst; and reducing the electrocatalyst at the electron conductor.

Another aspect is a method of generating electricity using the biofuel cells described above comprising oxidizing the fuel fluid at the anode or bioanode and reducing the oxidant at the cathode or biocathode; oxidizing the reduced form of the electron mediator during the reduction of the oxidant at the cathode or biocathode; and reducing the electron mediator at the electron conductor.

A further aspect of the invention is an enzyme immobilized in a hydrophobically modified polysaccharide. The hydrophobically modified polysaccharide being capable of immobilizing and stabilizing the enzyme and being permeable to a compound smaller than the enzyme.

Another aspect is an enzyme immobilized in a micellar hydrophobically modified polycationic polymer, the immobilized enzyme being more active than the enzyme when placed in a buffer solution.

A further aspect is an enzyme immobilized in a micellar hydrophobically modified polyanionic polymer, the immobilized enzyme being more active than the enzyme when placed in a buffer solution.

Yet another aspect is a micellar hydrophobically modified chitosan having at least about 10, 12, 14, 16, 18, 20, 22, 24, 26, 28, 30, 32, 34, 36, 38, 40, 42, 44, 46, 48% of the amine functionalities of the chitosan modified by hydrophobic groups.

Another aspect is a micellar hydrophobic redox mediator modified chitosan having a structure corresponding to Formula 1A

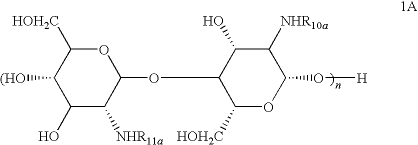

wherein n is an integer; R₁₀ₐ is independently hydrogen, or a hydrophobic redox mediator; and R₁₁ₐ is independently hydrogen, or a hydrophobic redox mediator.

### DESCRIPTION OF THE DRAWINGS

FIG. 1 shows representative fluorescence micrographs of hydrophobically modified chitosan in Ru(bpy)₃⁺².

FIG. 2 shows representative fluorescence micrographs of hydrophobically modified chitosan membranes soaked in FITC.

FIG. 3 shows the KD^{1/2} values for flux of caffeine through hydrophobically modified chitosan as a function of the alkyl chain length of the modifier and the solvent in which the polymer is resuspended.

FIG. 4 shows KD^{1/2} values for transport of Ru(bpy)₃⁺² through hydrophobically modified chitosan membranes.

FIG. 5 shows a single, functional bioanode or biocathode.

FIG. 6 shows a microfluidic biofuel cell.

FIG. 7(a)-(d) shows the procedure for forming a single microelectrode.

FIG. 8 shows a microfluidic biofuel cell stack.

FIG. 9 shows a series of power curves for a butyl-chitosan glucose dehydrogenase bioanode collected on various days from fabrication.

FIG. 10 is a power curve for a biofuel cell having a mediated bioanode (comprising tetrabutylammonium-modified Nafion® and NAD⁺-dependent alcohol dehydrogenase) and a direct electron transfer biocathode (comprising butyl-chitosan and bilirubin oxidase).

FIG. 11 is a power curve for a biofuel cell having a mediated bioanode (comprising butyl-chitosan and NAD⁺-dependent alcohol dehydrogenase) and a direct electron transfer biocathode (comprising butyl-chitosan and bilirubin oxidase).

FIG. 12 is a fluorescence micrograph of a low molecular weight alginate modified with tetrapentylammonium ions.

FIG. 13 is a schematic of an 1-cell comprising an air-breathing cathode.

### DETAILED DESCRIPTION OF THE INVENTION

The present invention is directed to bioanodes, biocathodes, biofuel cells, and enzyme immobilization materials comprising a hydrophobically modified polysaccharide, preferably, a hydrophobically modified chitosan or a hydrophobicall modified alginate. The hydrophobically modified polysaccharides form micellar structures having pores therein that are advantageously suited for immobilizing enzymes. Some of these hydrophobically modified polysaccharides are polycationic biopolymers that are biocompatible and are well suited for immobilizing enzymes in acidic to neutral environments (e.g., for enzymes that are active at pHs of about 5). In addition to its polycationic character, the hydrophobically modified polysaccharides can be modified with a variety of hydrophobic groups which can either alter the shape of the pores to fit the particular enzyme or alter the electronic characteristics of the enzyme immobilization material.

In yet a further embodiment, the bioelectrode assembly of the present invention has increased enzyme stability. For use in a biocathode or a bioanode, the immobilization material forms a barrier that provides mechanical and chemical stability. Thus, the enzyme is stabilized for a longer period than previously known. For purposes of the present invention, an enzyme is “stabilized” if it retains at least about 75% of its initial catalytic activity upon continuous operation in a biofuel cell for at least about 7 days to about 730 days.

### I. Biofuel Cell

Among the various aspects of the invention is a biofuel cell utilizing a fuel fluid to produce electricity via enzyme mediated redox reactions taking place at electrodes with immobilized enzymes therein. As in a standard electrochemical cell, the anode is the site for an oxidation reaction of a fuel fluid with a concurrent release of electrons. The electrons are directed from the anode through an electrical connector to some power consuming device. The electrons move through the device to another electrical connector, which transports the electrons to the biofuel cell's biocathode where the electrons are used to reduce an oxidant to produce water. In this manner, the biofuel cell of the present invention acts as an energy source (electricity) for an electrical load external thereto. To facilitate the fuel fluid's redox reactions, the electrodes comprise an electron conductor, an electron mediator, an electrocatalyst for the electron mediator, an enzyme, and an enzyme immobilization material.

In accordance with the invention, the electron mediator is a compound that can accept electrons or donate electrons. In a presently preferred biofuel cell, the oxidized form of the electron mediator reacts with the fuel fluid and the enzyme to produce the oxidized form of the fuel fluid and the reduced form of the electron mediator at the bioanode. Subsequently or concurrently, the reduced form of the electron mediator reacts with the oxidized form of the electrocatalyst to produce the oxidized form of the electron mediator and the reduced form of the electrocatalyst. The reduced form of the electrocatalyst is then oxidized at the bioanode and produces electrons to generate electricity. The redox reactions at the bioanode, except the oxidation of the fuel fluid, can be reversible, so the enzyme, electron mediator and electrocatalyst are not consumed. Optionally, these redox reactions can be irreversible if an electron mediator and/or an electrocatalyst is added to provide additional reactant.

Alternatively, an electron conductor and an enzyme can be used wherein an electron mediator in contact with the bioanode is able to transfer electrons between its oxidized and reduced forms at unmodified electrodes. If the electron mediator is able to transfer electrons between its oxidized and reduced forms at an unmodified bioanode, the subsequent reaction between the electrocatalyst and the electron mediator is not necessary and the electron mediator itself is oxidized at the bioanode to produce electrons and thus, electricity.

At the biocathode, electrons originating from the bioanode flow into the biocathode's electron conductor. There, the electrons combine with an oxidized form of an electrocatalyst, which is in contact with the electron conductor. This reaction produces a reduced form of the electrocatalyst, which in turn reacts with an oxidized form of an electron mediator to produce a reduced form of the electron mediator and an oxidized form of the electrocatalyst. Next, the reduced form of the electron mediator reacts with an oxidized form of the oxidant to produce an oxidized form of the electron mediator and water. In one embodiment, an enzyme immobilization material permeable to the oxidant is present, which comprises the electrocatalyst and, optionally, the electron mediator, and which is capable of immobilizing and stabilizing the enzyme.

In an alternative embodiment of the biocathode, there is no electrocatalyst present. In this embodiment, the electrons combine with an oxidized form of the electron mediator to produce a reduced form of the electron mediator. Then, the reduced form of the electron mediator reacts with an oxidized form of an oxidant to produce an oxidized form of the electron mediator and water. In one embodiment, an enzyme immobilization material permeable to the oxidant is present, which optionally comprises the electron mediator, and which is capable of immobilizing and stabilizing the enzyme.

The biofuel cell of the present invention comprises a biocathode and/or a bioanode. Generally, the bioanode comprises elements that effect the oxidation of fuel fluid whereby electrons are released and directed to an external electrical load. The resulting electrical current powers the electrical load, with electrons being subsequently directed to a biocathode where an oxidant is reduced and water is produced.

A. Biocathode

The biocathode in accordance with this invention comprises an electron conductor, an enzyme which is immobilized in an enzyme immobilization material, an electron mediator, and an electrocatalyst. In one embodiment, these components are adjacent to one another, meaning they are physically or chemically connected by appropriate means.

1. Electron Conductor

The electron conductor is a substance that conducts electrons. The electron conductor can be organic or inorganic in nature as long as it is able to conduct electrons through the material. The electron conductor can be a carbon-based material, stainless steel, stainless steel mesh, a metallic conductor, a semiconductor, a metal oxide, a modified conductor, or combinations thereof. In a preferred embodiment, the electron conductor is a carbon-based material.

Particularly suitable electron conductors are carbon-based materials. Exemplary carbon-based materials are carbon cloth, carbon paper, carbon screen printed electrodes, carbon paper (Toray), carbon paper (ELAT), carbon black (Vulcan XC-72, E-tek), carbon black, carbon powder, carbon fiber, single-walled carbon nanotubes, double-walled carbon nanotubes, multi-walled carbon nanotubes, carbon nanotubes arrays, diamond-coated conductors, glassy carbon and mesoporous carbon. In addition, other exemplary carbon-based materials are graphite, uncompressed graphite worms, delaminated purified flake graphite (Superior® graphite), high performance graphite and carbon powders (Formula BT™, Superior® graphite), highly ordered pyrolytic graphite, pyrolytic graphite and polycrystalline graphite. A preferred electron conductor (support membrane) is a sheet of carbon paper. Combinations of these carbon materials can be used.

In a further embodiment, the electron conductor can be made of a metallic conductor. Suitable electron conductors can be prepared from gold, platinum, iron, nickel, copper, silver, stainless steel, mercury, tungsten, and other metals suitable for electrode construction. In addition, electron conductors which are metallic conductors can be constructed of nanoparticles made of cobalt, carbon, and other suitable metals. Other metallic electron conductors can be silver-plated nickel screen printed electrodes.

In addition, the electron conductor can be a semiconductor. Suitable semiconductor materials include silicon and germanium, which can be doped with other elements. The semiconductors can be doped with phosphorus, boron, gallium, arsenic, indium or antimony, or a combination thereof.

Other electron conductors can be metal oxides, metal sulfides, main group compounds (i.e., transition metal compounds), and materials modified with electron conductors. Exemplary electron conductors of this type are nanoporous titanium oxide, tin oxide coated glass, cerium oxide particles, molybdenum sulfide, boron nitride nanotubes, aerogels modified with a conductive material such as carbon, solgels modified with conductive material such as carbon, ruthenium carbon aerogels, and mesoporous silicas modified with a conductive material such as carbon.

2. Electron Mediators

The electron mediator is a compound that can accept or donate electron(s). Stated another way, the electron mediator has an oxidized form that can accept electron(s) to form the reduced form, wherein the reduced form can also donate electron(s) to produce the oxidized form. The electron mediator is a compound that can diffuse into the immobilization material and/or be incorporated into the immobilization material.

In one embodiment, the diffusion coefficient of the electron mediator is maximized. Stated another way, mass transport of the reduced form of the electron mediator is as fast as possible. A fast mass transport of the electron mediator allows for a greater current and power density of the biofuel cell in which it is employed.

The biocathode's electron mediator can be a protein such as stellacyanin, a protein byproduct such as bilirubin, a sugar such as glucose, a sterol such as cholesterol, a fatty acid, a metalloprotein, or combinations thereof. The electron mediators can also be a coenzyme or substrate of an oxidase. In one preferred embodiment, the electron mediator at the biocathode is bilirubin.

3. Electrocatalyst for an Electron Mediator

Generally, the electrocatalyst is a substance that facilitates the release of electrons at the electron conductor by reducing the standard reduction potential of the electron mediator.

Typically, electrocatalysts according to the invention are organometallic cations with standard reduction potentials greater than +0.4 volts. Exemplary electrocatalysts are transition metal complexes, such as osmium, ruthenium, iron, nickel, rhodium, rhenium, and cobalt complexes. Preferred organometallic cations using these complexes comprise large organic aromatic ligands that allow for large electron self exchange rates Examples of large organic aromatic ligands include derivatives of 1,10-phenanthroline (phen), 2,2′-bipyridine (bpy) and 2,2′,2″-terpyridines (terpy), such as Ru(phen)₃⁺², Fe(phen)₃⁺², Ru(bpy)₃⁺², Os(bpy)₃⁺², and Os(terpy)₃⁺². In a preferred embodiment, the electrocatalyst is a ruthenium compound. Most preferably, the electrocatalyst at the biocathode is Ru(bpy)₃⁺² (represented by Formula 2).

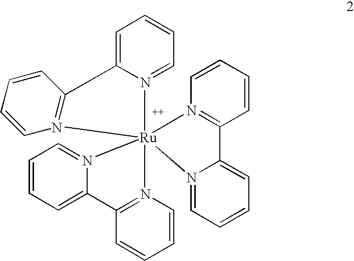

The electrocatalyst is present in a concentration that facilitates the efficient transfer of electrons. Preferably, the electrocatalyst is present at a concentration that makes the enzyme immobilization material conduct electrons. Particularly, the electrocatalyst is present at a concentration of from about 10 mM to about 3 M, more preferably from about 250 mM to about 2.25 M, still more preferably from about 500 mM to about 2 M, and most preferably from about 1.0 M to about 1.5 M.

4. Enzyme

In accordance with the invention, an enzyme reduces an oxidant at the biocathode. Generally, naturally-occurring enzymes, man-made enzymes, artificial enzymes and modified naturally-occurring enzymes can be utilized. In addition, engineered enzymes that have been engineered by natural or directed evolution can be used. Stated another way, an organic or inorganic molecule that mimics an enzyme's properties can be used in an embodiment of the present invention.

Specifically, exemplary enzymes for use in a biocathode are oxidoreductases. Potential oxidoreductases include laccases and oxidases, such as glucose oxidase, alcohol-based oxidases, and cholesterol-based oxidases. In a preferred embodiment, the enzyme is a peroxidase or oxygen oxidoreductase, which catalyze the reduction hydrogen peroxide and oxygen, respectively. Exemplary oxygen oxidoreductases include laccase, cytochrome c oxidase, bilirubin oxidase and peroxidase. More preferably, the enzyme is an oxygen oxidoreductase having an optimum activity at a pH between about 6.5 and about 7.5. An oxidoreductase having an optimum activity at a pH from about 6.5 to about 7.5 is advantageous for applications directed to a physiological environment, such as a plant or a human or animal body. Most preferably, the enzyme is a bilirubin oxidase.

5. Enzyme Immobilization Material

An enzyme immobilization material is utilized in the biofuel cell at the bioanode and/or the biocathode. In one embodiment, the bioanode's enzyme immobilization material is permeable to the fuel fluid and immobilizes and stabilizes the enzyme. The immobilization material is permeable to the fuel fluid so the oxidation reaction of the fuel at the bioanode can be catalyzed by the immobilized enzyme.

Generally, an enzyme is used to catalyze redox reactions at the biocathode and/or the bioanode. In a bioanode and/or biocathode according to this invention, an enzyme is immobilized in an enzyme immobilization material that both immobilizes and stabilizes the enzyme. Typically, a free enzyme in solution loses its catalytic activity within a few hours to a few days, whereas a properly immobilized and stabilized enzyme can retain its catalytic activity for at least about 7 days to about 730 days. The retention of catalytic activity is defined as the enzyme having at least about 75% of its initial activity, which can be measured by chemiluminescence, electrochemical, UV-Vis, radiochemical, or fluorescence assay. The enzyme retains at least about 75% of its initial activity while the biofuel cell is continually producing electricity for at least about 7 days to about 730 days.

An immobilized enzyme is an enzyme that is physically confined in a certain region of the enzyme immobilization material while retaining its catalytic activity. There are a variety of methods for enzyme immobilization, including carrier-binding, cross-linking and entrapping. Carrier-binding is the binding of enzymes to water-insoluble carriers. Cross-linking is the intermolecular cross-linking of enzymes by bifunctional or multifunctional reagents. Entrapping is incorporating enzymes into the lattices of a semipermeable material. The particular method of enzyme immobilization is not critically important, so long as the enzyme immobilization material (1) immobilizes the enzyme, (2) stabilizes the enzyme, and (3) is permeable to the fuel fluid or oxidant.

With reference to the enzyme immobilization material's permeability to the fuel fluid or oxidant and the immobilization of the enzyme, in one embodiment, the material is permeable to a compound that is smaller than an enzyme. Stated another way, the enzyme immobilization material allows the movement of the fuel fluid or oxidant compound through it so the compound can contact the enzyme. The enzyme immobilization material can be prepared in a manner such that it contains internal pores, channels, openings or a combination thereof, which allow the movement of the compound throughout the enzyme immobilization material, but which constrain the enzyme to substantially the same space within the enzyme immobilization material. Such constraint allows the enzyme to retain its catalytic activity. In various preferred embodiments, the enzyme is confined to a space that is substantially the same size and shape as the enzyme, wherein the enzyme retains substantially all of its catalytic activity. The pores, channels, or openings have physical dimensions that satisfy the above requirements and depend on the size and shape of the specific enzyme to be immobilized.

In one embodiment, the enzyme is preferably located within a pore of the enzyme immobilization material and the compound travels in and out of the enzyme immobilization material through transport channels. The relative size of the pores and transport channels can be such that a pore is large enough to immobilize an enzyme, but the transport channels are too small for the enzyme to travel through them. Further, a transport channel preferably has a diameter of at least about 10 nm. In still another embodiment, the pore diameter to transport channel diameter ratio is at least about 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1 or more. In yet another embodiment, preferably, a transport channel has a diameter of at least about 10 nm and the pore diameter to transport channel diameter ratio is at least about 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1 or more.

With respect to the stabilization of the enzyme, the enzyme immobilization material provides a chemical and mechanical barrier to prevent or impede enzyme denaturation. To this end, the enzyme immobilization material physically confines the enzyme, preventing the enzyme from unfolding. The process of unfolding an enzyme from a folded three-dimensional structure is one mechanism of enzyme denaturation. In one embodiment, the immobilization material, preferably, stabilizes the enzyme so that the enzyme retains its catalytic activity for at least about 7 days to about 730 days. The retention of catalytic activity is defined by the number of days that the enzyme retains at least about 75% of its initial activity while continually producing electricity as part of a biofuel cell. The enzyme activity can be measured by chemiluminescence, electrochemical, UV-Vis, radiochemical or fluorescence assay wherein the intensity of the property is measured at an initial time. Typically, a fluorescence assay is used to measure the enzyme activity. A free enzyme in solution loses its catalytic activity within hours to a few days. Thus, the immobilization of the enzyme provides a significant advantage in stability. In another embodiment, preferably, the immobilized enzyme retains at least about 75% of its initial catalytic activity for at least about 5, 10, 15, 20, 25, 30, 45, 60, 75, 90, 105, 120, 150, 180, 210, 240, 270, 300, 330, 365, 400, 450, 500, 550, 600, 650, 700, 730 days or more, preferably retaining at least about 80%, 85%, 90%, 95% or more of its initial catalytic activity for at least about 5, 10, 15, 20, 25, 30, 45, 60, 75, 90, 105, 120, 150, 180, 210, 240, 270, 300, 330, 365, 400, 450, 500, 550, 600, 650, 700, 730 days or more.

In some of the embodiments, the enzyme immobilization material has a micellar or inverted micellar structure. Generally, the molecules making up a micelle are amphipathic, meaning they contain a polar, hydrophilic group and a nonpolar, hydrophobic group. The molecules can aggregate to form a micelle, where the polar groups are on the surface of the aggregate and the hydrocarbon, nonpolar groups are sequestered inside the aggregate. Inverted micelles have the opposite orientation of polar groups and nonpolar groups. The amphipathic molecules making up the aggregate can be arranged in a variety of ways so long as the polar groups are in proximity to each other and the nonpolar groups are in proximity to each other. Also, the molecules can form a bilayer with the nonpolar groups pointing toward each other and the polar groups pointing away from each other. Alternatively, a brayer can form wherein the polar groups can point toward each other in the bilayer, while the nonpolar groups point away from each other.

In one preferred embodiment, the micellar enzyme immobilization material is a modified perfluoro sulfonic acid-PTFE copolymer (or modified perfluorinated ion exchange polymer)(modified Nafion® or modified Flemion®) membrane. The perfluorinated ion exchange polymer membrane is modified with a hydrophobic cation that is larger than the ammonium (NH₄⁺) ion. The hydrophobic cation serves the dual function of (1) dictating the membrane's pore size and (2) acting as a chemical buffer to help maintain the pore's pH level, both of which stabilize the enzyme.

With regard to the first function of the hydrophobic cation, mixture-casting a perfluoro sulfonic acid-PTFE copolymer (or perfluorinated ion exchange polymer) with a hydrophobic cation to produce a modified perfluoro sulfonic acid-PTFE copolymer (or modified perfluorinated ion exchange polymer)(Nafion® or Flemion®) membrane provides an enzyme immobilization material wherein the pore size is dependent on the size of the hydrophobic cation. Accordingly, the larger the hydrophobic cation, the larger the pore size. This function of the hydrophobic cation allows the pore size to be made larger or smaller to fit a specific enzyme by varying the size of the hydrophobic cation.

Regarding the second function of the hydrophobic cation, the properties of the perfluoro sulfonic acid-PTFE copolymer (or perfluorinated ion exchange polymer) membrane are altered by exchanging the hydrophobic cation for protons as the counterion to the —SO₃⁻ groups on the perfluoro sulfonic acid-PTFE copolymer (or anions on the perfluorinated ion exchange polymer) membrane. This change in counterion provides a buffering effect on the pH because the hydrophobic cation has a much greater affinity for the —SO₃⁻ sites than protons do. This buffering effect of the membrane causes the pH of the pore to remain substantially unchanged with changing solution pH; stated another way, the pH of the pore resists changes in the solution's pH. In addition, the membrane provides a mechanical barrier, which further protects the immobilized enzymes. In order to prepare a modified perfluoro sulfonic acid-PTFE copolymer (or perfluorinated ion exchange polymer) membrane, the first step is to cast a suspension of perfluoro sulfonic acid-PTFE copolymer (or perfluorinated ion exchange polymer), particularly Nafion®, with a solution of the hydrophobic cations to form a membrane. The excess hydrophobic cations and their salts are then extracted from the membrane, and the membrane is re-cast. Upon re-casting, the membrane contains the hydrophobic cations in association with the —SO₃⁻ sites of the perfluoro sulfonic acid-PTFE copolymer (or perfluorinated ion exchange polymer) membrane. Removal of the salts of the hydrophobic cation from the membrane results in a more stable and reproducible membrane since the excess salts can become trapped in the pore or cause voids in the membrane.

In one embodiment, a modified Nafion® membrane is prepared by casting a suspension of Nafion® polymer with a solution of a salt of a hydrophobic cation such as quaternary ammonium bromide. Excess quaternary ammonium bromide or hydrogen bromide are removed from the membrane before it is re-cast to form the salt-extracted membrane. Salt extraction of membranes retains the presence of the quaternary ammonium cations at the sulfonic acid exchange sites, but eliminates complications from excess salt that may be trapped in the pore or may cause voids in the equilibrated membrane. The chemical and physical properties of the salt-extracted membranes have been characterized by voltammetry, ion exchange capacity measurements, and fluorescence microscopy before enzyme immobilization. Exemplary hydrophobic cations are ammonium-based cations, quaternary ammonium cations, alkyltrimethylammonium cations, alkyltrimethylammonium cations, organic cations, phosphonium cations, triphenylphosphonium, pyridinium cations, imidazolium cations, hexadecylpyridinium, ethidium, viologens, methyl viologen, benzyl viologen, bis(triphenylphosphine)iminium, metal complexes, bipyridyl metal complexes, phenanthroline-based metal complexes, [Ru(bipyridine)₃]²⁺ and [Fe(phenanthroline)₃]³⁺.

In one preferred embodiment, the hydrophobic cations are ammonium-based cations. In particular, the hydrophobic cations are quaternary ammonium cations. In another embodiment, the quaternary ammonium cations are represented by Formula 4:

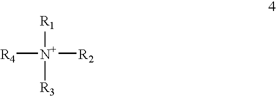

wherein R₁, R₂, R₃, and R₄ are independently hydrogen, hydrocarbyl, substituted hydrocarbyl, or heterocyclo wherein at least one of R₁, R₂, R₃, and R₄ is other than hydrogen. In a further embodiment, preferably, R₁, R₂, R₃, and R₄ are independently hydrogen, methyl, ethyl, propyl, butyl, pentyl, hexyl, heptyl, octyl, nonyl, decyl, undecyl, dodecyl, tridecyl or tetradecyl wherein at least one of R₁, R₂, R₃, and R₄ is other than hydrogen. In still another embodiment, R₁, R₂, R₃, and R₄ are the same and are methyl, ethyl, propyl, butyl, pentyl or hexyl. In yet another embodiment, preferably, R₁, R₂, R₃, and R₄ are butyl. Preferably, the quaternary ammonium cation is tetrapropylammonium (T3A), tetrapentylammonium (T5A), tetrahexylammonium (T6A), tetraheptylammonium (T7A), trimethyl icosylammonium (TMICA), trimethyloctyldecylammonium (TMODA), trimethylhexyldecylammonium (TMHDA), trimethyltetradecylammonium (TMTDA), trimethyloctylammonium (TMOA), trimethyldodecylammonium (TMDDA), trimethyldecylammonium (TMDA), trimethylhexylammonium (TMHA), tetrabutylammonium (TBA), triethylhexylammonium (TEHA), and combinations thereof.

In other various embodiments, exemplary micellar or inverted micellar enzyme immobilization materials are, hydrophobically modified polysaccharides, these polysaccharides are selected from chitosan, cellulose, chitin, starch, amylose, alginate, and combinations thereof. In various embodiments, the micellar or inverted micellar enzyme immobilization materials are polycationic polymers, particularly, hydrophobically modified chitosan. Chitosan is a poly[β-(1-4)-2-amino-2-deoxy-D-glucopyranose]. Chitosan is typically prepared by deacetylation of chitin (a poly[β-(1-4)-2-acetamido-2-deoxy-D-glucopyranose]). The typical commercial chitosan has approximately 85% deacetylation. These deacetylated or free amine groups can be further functionalized with hydrocarbyl, particularly, alkyl groups. Thus, in various embodiments, the micellar hydrophobically modified chitosan corresponds to the structure of Formula 1

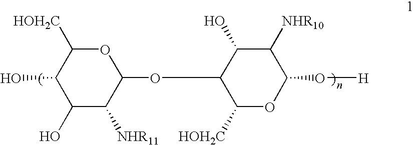

wherein n is an integer; R₁₀ is independently hydrogen, hydrocarbyl, substituted hydrocarbyl, or a hydrophobic redox mediator; and R₁₁ is independently hydrogen, hydrocarbyl, substituted hydrocarbyl, or a hydrophobic redox mediator. In certain embodiments of the invention, n is an integer that gives the polymer a molecular weight of from about 21,000 to about 500,000; preferably, from about 90,000 to about 500,000; more preferably, from about 150,000 to about 350,000; more preferably, from about 225,000 to about 275,000. In many embodiments, R₁₀ is independently hydrogen or alkyl and R₁₁ is independently hydrogen or alkyl. Further, R₁₀ is independently hydrogen or hexyl and R₁₁ is independently hydrogen or hexyl. Alternatively, R₁₀ is independently hydrogen or octyl and R₁₁ is independently hydrogen or octyl.

In other various embodiments, the micellar hydrophobically modified chitosan is a micellar hydrophobic redox mediator modified chitosan corresponding to Formula 1A

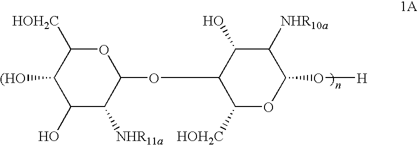

wherein n is an integer; R₁₀ₐ is independently hydrogen, or a hydrophobic redox mediator; and R₁₁ₐ is independently hydrogen, or a hydrophobic redox mediator.

Further, in various embodiments, the micellar hydrophobically modified chitosan is a modified chitosan or redox mediator modified chitosan corresponding to Formula 1B

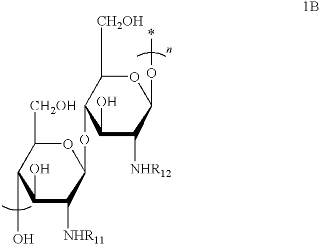

wherein R₁₁, R₁₂, and n are defined as in connection with Formula 1. In some embodiments, R₁₁ and R₁₂ are independently hydrogen or straight or branched alkyl; preferably, hydrogen, butyl, pentyl, hexyl, heptyl, octyl, nonyl, decyl, undecyl, or dodecyl. In various embodiments, R₁₁ and R₁₂ are independently hydrogen, butyl, or hexyl.

The micellar hydrophobically modified chitosans can be modified with hydrophobic groups to varying degrees. The degree of hydrophobic modification is determined by the percentage of free amine groups that are modified with hydrophobic groups as compared to the number of free amine groups in the unmodified chitosan. The degree of hydrophobic modification can be estimated from an acid-base titration and/or nuclear magnetic resonance (NMR), particularly ¹H NMR, data. This degree of hydrophobic modification can vary widely and is at least about 1, 2, 4, 6, 8, 10, 12, 14, 16, 18, 20, 25, 30, 32, 24, 26, 28, 40, 42, 44, 46, 48%, or more. Preferably, the degree of hydrophobic modification is from about 10% to about 45%; from about 10% to about 35%; from about 20% to about 35%; or from about 30% to about 35%.

In other various embodiments, the hydrophobic redox mediator of Formula 1A is a transition metal complex of osmium, ruthenium, iron, nickel, rhodium, rhenium, or cobalt with 1,10-phenanthroline (phen), 2,2′-bipyridine (bpy) or 2,2′,2″-terpyridine (terpy), methylene green, methylene blue, poly(methylene green), poly(methylene blue), luminol, nitro-fluorenone derivatives, azines, osmium phenanthrolinedione, catechol-pendant terpyridine, toluene blue, cresyl blue, nile blue, neutral red, phenazine derivatives, tionin, azure A, azure B, toluidine blue O, acetophenone, metallophthalocyanines, nile blue A, modified transition metal ligands, 1,10-phenanthroline-5,6-dione, 1,10-phenanthroline-5,6-diol, [Re(phen-dione)(CO)₃Cl], [Re(phen-dione)₃](PF₆)₂, poly(metallophthalocyanine), poly(thionine), quinones, diimines, diaminobenzenes, diaminopyridines, phenothiazine, phenoxazine, toluidine blue, brilliant cresyl blue, 3,4-dihydroxybenzaldehyde, poly(acrylic acid), poly(azure I), poly(nile blue A), polyaniline, polypyridine, polypyrole, polythiophene, poly(thieno[3,4-b]thiophene), poly(3-hexylthiophene), poly(3,4-ethylenedioxypyrrole), poly(isothianaphthene), poly(3,4-ethylenedioxythiophene), poly(difluoroacetylene), poly(4 dicyanomethylene-4H-cyclopenta[2,1-b;3,4-b′]dithiophene), poly(3-(4-fluorophenyl)thiophene), poly(neutral red), or combinations thereof.

Preferably, the hydrophobic redox mediator is Ru(phen)₃⁺², Fe(phen)₃⁺², Os(phen)₃⁺², Co(phen)₃⁺², Cr(phen)₃⁺², Ru(bpy)₃⁺², Os(bpy)₃⁺², Fe(bpy)₃⁺², Co(bpy)₃⁺², Cr(bpy)₃⁺², Os(terpy)₃⁺², Ru(bpy)₂(4-methyl-4′-(6-hexyl)-2,2′-bipyridine)⁺², Co(bpy)₂(4-methyl-4′-(6-hexyl)-2,2′-bipyridine)⁺², Cr(bpy)₂(4-methyl-4′-(6-hexyl)-2,2′-bipyridine)⁺², Fe(bpy)₂(4-methyl-4′-(6-hexyl)-2,2′-bipyridine)⁺², Os(bpy)₂(4-methyl-4′-(6-hexyl)-2,2′-bipyridine)⁺², or combinations thereof. More preferably, the hydrophobic redox mediator is Ru(bpy)₂(4-methyl-4′-(6-hexyl)-2,2′-bipyridine)⁺², Co(bpy)₂(4-methyl-4′-(6-hexyl)-2,2′-bipyridine)⁺², Cr(bpy)₂(4-methyl-4′-(6-hexyl)-2,2′-bipyridine)⁺², Fe(bpy)₂(4-methyl-4′-(6-hexyl)-2,2′-bipyridine)⁺², Os(bpy)₂(4-methyl-4′-(6-hexyl)-2,2′-bipyridine)⁺², or combinations thereof. In various preferred embodiments, the hydrophobic redox mediator is Ru(bpy)₂(4-methyl-4′-(6-hexyl)-2,2′-bipyridine)⁺².

For the immobilization material having a hydrophobic redox mediator as the modifier, the hydrophobic redox mediator is typically covalently bonded to the chitosan or polysaccharide backbone. Typically, in the case of chitosan, the hydrophobic redox mediator is covalently bonded to one of the amine functionalities of the chitosan through a —N—C— bond. In the case of metal complex redox mediators, the metal complex is attached to the chitosan through an —N—C— bond from a chitosan amine group to an alkyl group attached to one or more of the ligands of the metal complex. A structure corresponding to Formula 1C is an example of a metal complex attached to a chitosan

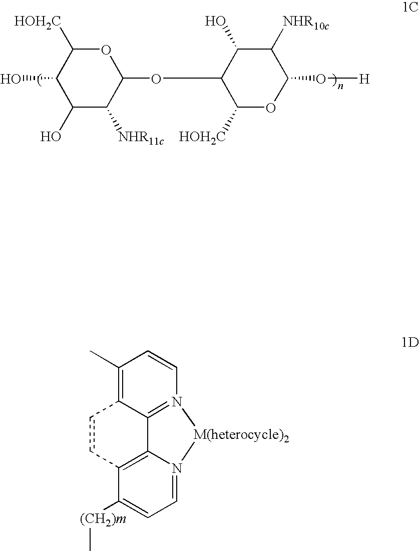

wherein n is an integer; R_{10c} is independently hydrogen or a structure corresponding to Formula 1D; R_{11c} is independently hydrogen or a structure corresponding to Formula 1D; m is an integer from 0 to 10; and M is Ru, Os, Fe, Cr, or Co.

The hydrophobic group used to modify chitosan serves the dual function of (1) dictating the immobilization material's pore size and (2) modifying the chitosan's electronic environment to maintain an acceptable pore environment, both of which stabilize the enzyme. With regard to the first function of the hydrophobic group, hydrophobically modifying chitosan produces an enzyme immobilization material wherein the pore size is dependent on the size of the hydrophobic group. Accordingly, the size, shape, and extent of the modification of the chitosan with the hydrophobic group affects the size and shape of the pore. This function of the hydrophobic group allows the pore size to be made larger or smaller or a different shape to fit a specific enzyme by varying the size and branching of the hydrophobic group.

Regarding the second function of the hydrophobic cation, the properties of the hydrophobically modified chitosan membranes are altered by modifying chitosan with hydrophobic groups. This hydrophobic modification of chitosan affects the pore environment by increasing the number of available exchange sites to proton. In addition to affecting the pH of the material, the hydrophobic modification of chitosan provides a membrane that is a mechanical barrier, which further protects the immobilized enzymes.

Table 1 shows the number of available exchange sites to proton for the hydrophobically modified chitosan membrane.

TABLE 1
Number of available exchange sites to
proton per gram of chitosan polymer

| | Exchange sites per gram |
|---|---|
| Membrane | (×10⁻⁴ mol SO₃/g) |
| Chitosan | 10.5 ± 0.8 |
| Butyl Modified | 226 ± 21 |
| Hexyl Modified | 167 ± 45 |
| Octyl Modified | 529 ± 127 |
| Decyl Modified | 483 ± 110 |

Further, such polycationic polymers are capable of immobilizing enzymes and increasing the activity of enzymes immobilized therein as compared to the activity of the same enzyme in a buffer solution. In various embodiments, the polycationic polymers are hydrophobically modified polysaccharides, particularly, hydrophobically modified chitosan. For example, for the hydrophobic modifications noted, the enzyme activities for glucose oxidase were measured using the procedure in Example 5. The highest enzyme activity was observed for glucose oxidase in a hexyl modified chitosan suspended in t-amyl alcohol. These immobilization membranes showed a 2.53 fold increase in glucose oxidase enzyme activity over enzyme in buffer. Table 2 details the glucose oxidase activities for a variety of hydrophobically modified chitosans.

TABLE 2
Glucose oxidase enzyme activity for modified chitosans

| | Enzyme Activity |
|---|---|
| Membrane/Solvent | (Units/gm) |
| Buffer | 103.61 ± 3.15 |
| UNMODIFIED CHITOSAN | 214.86 ± 10.23 |

HEXYL CHITOSAN

| | |
|---|---|
| Chloroform | 248.05 ± 12.62 |
| t-amyl alcohol | 263.05 ± 7.54 |
| 50% acetic acid | 118.98 ± 6.28 |

DECYL CHITOSAN

| | |
|---|---|
| Chloroform | 237.05 ± 12.31 |
| t-amyl alcohol | 238.05 ± 10.02 |
| 50% acetic acid | 3.26 ± 2.82 |

OCTYL CHITOSAN

| | |
|---|---|
| Chloroform | 232.93 ± 7.22 |
| t-amyl alcohol | 245.75 ± 9.77 |
| 50% acetic acid | 127.55 ± 11.98 |

BUTYL CHITOSAN

| | |
|---|---|
| Chloroform | 219.15 ± 9.58 |
| t-amyl alcohol | 217.10 ± 6.55 |
| 50% acetic acid | 127.65 ± 3.02 |

To prepare the hydrophobically modified chitosans of the invention having an alkyl group as a modifier, a chitosan gel was suspended in acetic acid followed by addition of an alcohol solvent. To this chitosan gel was added an aldehyde (e.g., butanal, hexanal, octanal, or decanal), followed by addition of sodium cyanoborohydride. The resulting product was separated by vacuum filtration and washed with an alcohol solvent. The modified chitosan was then dried in a vacuum oven at 40° C., resulting a flaky white solid.

To prepare a hydrophobically modified chitosan of the invention having a redox mediator as a modifier, a redox mediator ligand was derivatized by contacting 4,4′-dimethyl-2,2′-bipyridine with lithium diisopropylamine followed by addition of a dihaloalkane to produce 4-methyl-4′-(6-haloalkyl)-2,2′-bipyridine. This ligand was then contacted with Ru(bipyridine)₂Cl₂ hydrate in the presence of an inorganic base and refluxed in a water-alcohol mixture until the Ru(bipyridine)₂Cl₂ was depleted. The product was then precipitated with ammonium hexafluorophosphate, or optionally a sodium or potassium perchlorate salt, followed by recrystallization. The derivatized redox mediator (Ru(bipyridine)₂(4-methyl-4′-(6-bromohexyl)-2,2′-bipyridine)⁺²) was then contacted with deacetylated chitosan and heated. The redox mediator modified chitosan was then precipitated and recrystallized.

The hydrophobically modified chitosan membranes have advantageous insolubility in ethanol. For example, the chitosan enzyme immobilization materials described above generally are functional to immobilize and stabilize the enzymes in solutions having up to greater than about 99 wt. % or 99 volume % ethanol. In various embodiments, the chitosan immobilization material is functional in solutions having 15, 20, 25, 30, 35, 40, 45, 50, 55, 60, 65, 70, 75, 80, 85, 90, 95 or more wt. % or volume % ethanol.

In other embodiments, the micellar or inverted micellar enzyme immobilization materials are polyanionic polymers, such as hydrophobically modified polysaccharides, particularly, hydrophobically modified alginate. Alginates are linear unbranched polymers containing β-(1-4)-linked D-mannuronic acid and α-(1-4)-linked L-guluronic acid residues. In the unprotonated form, β-(1-4)-linked D-mannuronic acid corresponds to the structure of Formula 3A

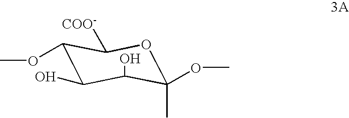

and in the unprotonated form, α-(1-4)-linked L-guluronic acid corresponds to the structure of Formula 3B

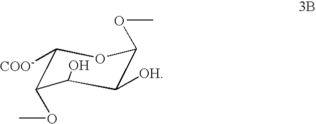

Alginate is a heterogeneous polymer consisting of polymer blocks of mannuronic acid residues and polymer blocks of guluronic acid residues.

Alginate polymers can be modified in various ways One type is alginate modified with a hydrophobic cation that is larger than the ammonium (NH₄⁺) ion. The hydrophobic cation serves the dual function of (1) dictating the polymer's pore size and (2) acting as a chemical buffer to help maintain the pore's pH level, both of which stabilize the enzyme. With regard to the first function of the hydrophobic cation, modifying alginate with a hydrophobic cation produces an enzyme immobilization material wherein the pore size is dependent on the size of the hydrophobic cation. Accordingly, the size, shape, and extent of the modification of the alginate with the hydrophobic cation affects the size and shape of the pore. This function of the hydrophobic cation allows the pore size to be made larger or smaller or a different shape to fit a specific enzyme by varying the size and branching of the hydrophobic cation.

Regarding the second function of the hydrophobic cation, the properties of the alginate polymer are altered by exchanging the hydrophobic cation for protons as the counterion to the —CO₂⁻ groups on the alginate. This change in counterion provides a buffering effect on the pH because the hydrophobic cation has a much greater affinity for the —CO₂⁻ sites than protons do. This buffering effect of the alginate membrane causes the pH of the pore to remain substantially unchanged with changing solution pH; stated another way, the pH of the pore resists changes in the solution's pH. In addition, the alginate membrane provides a mechanical barrier, which further protects the immobilized enzymes.

In order to prepare a modified alginate membrane, the first step is to cast a suspension of alginate polymer with a solution of the hydrophobic cation to form a membrane. The excess hydrophobic cations and their salts are then extracted from the membrane, and the membrane is re-cast. Upon re-casting, the membrane contains the hydrophobic cations in association with —CO₂⁻ sites of the alginate membrane. Removal of the salts of the hydrophobic cation from the membrane results in a more stable and reproducible membrane since the excess salts can become trapped in the pore or cause voids in the membrane.

In one embodiment, a modified alginate membrane is prepared by casting a suspension of alginate polymer with a solution of a salt of a hydrophobic cation such as quaternary ammonium bromide. Excess quaternary ammonium bromide or hydrogen bromide are removed from the membrane before it is re-cast to form the salt-extracted membrane. Salt extraction of membranes retains the presence of the quaternary ammonium cations at the carboxylic acid exchange sites, but eliminates complications from excess salt that may be trapped in the pore or may cause voids in the equilibrated membrane. Exemplary hydrophobic cations are ammonium-based cations, quaternary ammonium cations, alkyltrimethylammonium cations, alkyltriethylammonium cations, organic cations, phosphonium cations, triphenylphosphonium, pyridinium cations, imidazolium cations, hexadecylpyridinium, ethidium, viologens, methyl viologen, benzyl viologen, bis(triphenylphosphine)iminium, metal complexes, bipyridyl metal complexes, phenanthroline-based metal complexes, [Ru(bipyridine)₃]²⁺ and [Fe(phenanthroline)₃]³⁺.

In one preferred embodiment, the hydrophobic cations are ammonium-based cations. In particular, the hydrophobic cations are quaternary ammonium cations. In another embodiment, the quaternary ammonium cations are represented by Formula 4:

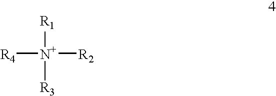

wherein R₁, R₂, R₃, and R₄ are independently hydrogen, hydrocarbyl, substituted hydrocarbyl, or heterocyclo wherein at least one of R₁, R₂, R₃, and R₄ is other than hydrogen. In a further embodiment, preferably, R₁, R₂, R₃, and R₄ are independently hydrogen, methyl, ethyl, propyl, butyl, pentyl, hexyl, heptyl, octyl, nonyl, decyl, undecyl, dodecyl, tridecyl or tetradecyl wherein at least one of R₁, R₂, R₃, and R₄ is other than hydrogen. In still another embodiment, R₁, R₂, R₃, and R₄ are the same and are methyl, ethyl, propyl, butyl, pentyl or hexyl. In yet another embodiment, preferably, R₁, R₂, R₃, and R₄ are butyl. Preferably, the quaternary ammonium cation is tetrapropylammonium (T3A), tetrapentylammonium (T5A), tetrahexylammonium (T6A), tetraheptylammonium (T7A), trimethylicosylammonium (TMICA), trimethyloctyidecylammonium (TMODA), trimethylhexyldecylammonium (TMHDA), trimethyltetradecylammonium (TMTDA), trimethyloctylammonium (TMOA), trimethyldodecylammonium (TMDDA), trimethyldecylammonium (TMDA), trimethylhexylammonium (TMHA), tetrabutylammonium (TBA), triethylhexylammonium (TEHA), and combinations thereof.

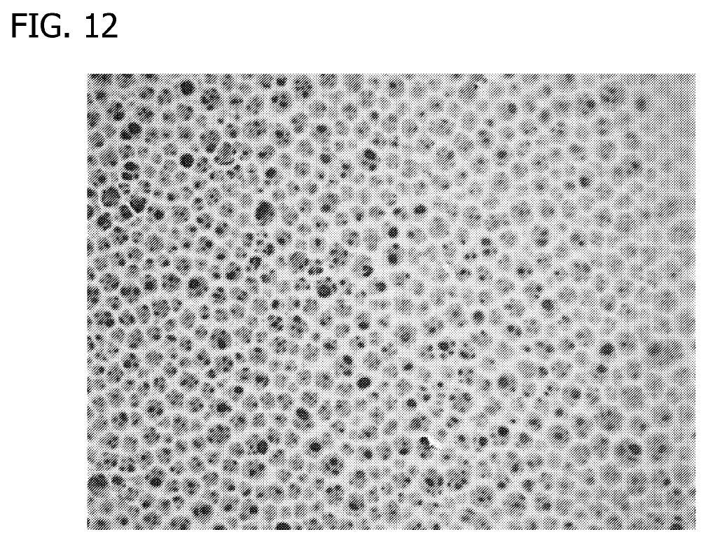

The pore characteristics were studied and the results for one hydrophobically modified alginate membrane are shown in FIG. 12. The pore structure of this membrane is ideal for enzyme immobilization, because the pores are hydrophobic, micellar in structure, buffered to external pH change, and have high pore interconnectivity.

In another experiment, ultralow molecular weight alginate and dodecylamine were placed in 25% ethanol and refluxed to produce a dodecyl-modified alginate by amidation of the carboxylic acid groups. Various alkyl amines can be substituted for the dodecylamine to produce alkyl-modified alginate having a C₄-C₁₆ alkyl group attached to varying numbers of the reactive carboxylic acid groups of the alginate structure. In various embodiments, at least about 1, 2, 4, 6, 8, 10, 12, 14, 16, 18, 20, 22, 24, 26, 28, 30, 32, 34, 36, 38, 40, 42, 44, 46, 48%, or more of the carboxylic acid groups react with the alkylamine.

The hydrophobically modified alginate membranes have advantageous insolubility in ethanol. For example, the alginate enzyme immobilization materials described above generally are functional to immobilize and stabilize the enzymes in solutions having at least about 25 wt. % or 25 volume % ethanol. In various embodiments, the alginate immobilization material is functional in solutions having 25, 30, 35, 40, 45, 50, 55, 60, 65, 70, 75, 80, 85, 90 or more wt. % or volume % ethanol.

6. Biocathode Embodiments

Various biocathodes can be incorporated into the biofuel cells of the present invention. For example, such biocathodes are described in U.S. patent application Ser. No. 10/931,147 (published as U.S. Patent Application Publication No. 2005/0095466), herein incorporated by reference in its entirety.

B. Bioanode

In one embodiment, the bioanode comprises an electron conductor and an enzyme which is immobilized in an enzyme immobilization material. In another embodiment, the bioanode optionally further comprises an electrocatalyst for an electron mediator. An electrocatalyst can be absent from the bioanode when the bioanode contacts an electron mediator that is capable of undergoing a reversible redox reaction at the electron conductor. The above-identified components of the bioanode are adjacent to one another; meaning they are physically or chemically connected by appropriate means. As the components are generally the same as the biocathode components, the following discussion concerns the differences in composition of the respective elements and differences in function, where appropriate.

1. Electron Conductor

As with the biocathode, the bioanode's electron conductor can be organic or inorganic in nature as long as it is able to conduct electrons through the material. In one embodiment, the bioanode electron conductor is carbon paper.

2. Electron Mediators

The bioanode electron mediator serves to accept or donate electron(s), readily changing from oxidized to reduced forms. The electron mediator is a compound that can diffuse into the immobilization material and/or be incorporated into the immobilization material. As with the biocathode, it is preferred that the electron mediator's diffusion coefficient is maximized.

Exemplary electron mediators are nicotinamide adenine dinucleotide (NAD⁺), flavin adenine dinucleotide (FAD), nicotinamide adenine dinucleotide phosphate (NADP), pyrroloquinoline quinone (PQQ), equivalents of each, and combinations thereof. Other exemplary electron mediators are phenazine methosulfate, dichlorophenol indophenol, short chain ubiquinones, potassium ferricyanide, a protein, a metalloprotein, stellacyanin, and combinations thereof. In one preferred embodiment, the electron mediator at the bioanode is NAD⁺.

Where the electron mediator cannot undergo a redox reaction at the electron conductor by itself, the bioanode comprises an electrocatalyst for an electron mediator which facilitates the release of electrons at the electron conductor. Alternatively, a reversible redox couple that has a standard reduction potential of 0.0V±0.5 V is used as the electron mediator. Stated another way, an electron mediator that provides reversible electrochemistry on the electron conductor surface can be used. The electron mediator is coupled with a naturally occurring enzyme that is dependent on that electron mediator, an enzyme modified to be dependent on that electron mediator, or a synthetic enzyme that is dependent on that electron mediator. Examples of electron mediators that provide reversible electrochemistry on the electron conductor surface is pyrroloquinoline quinone (PQQ), phenazine methosulfate, dichlorophenol indophenol, short chain ubiquinones and potassium ferricyanide. In this embodiment, the preferred electron mediator utilized with the bioanode is PQQ. Due to the capability of the electron mediator to provide reversible electrochemistry at the electron conductor surface, no electrocatalyst is necessary to catalyze the redox reaction in this embodiment.

Preferred compounds that are substrates for electrocatalysis by the redox polymer of the bioanode include reduced adenine dinucleotides, such as NADH, FADH₂ and NADPH.

3. Electrocatalyst for an Electron Mediator

Generally, the electrocatalyst is a substance that facilitates the release of electrons at the electron conductor. Stated another way, the electrocatalyst improves the kinetics of a reduction or oxidation of an electron mediator so the electron mediator reduction or oxidation can occur at a lower standard reduction potential. The electrocatalyst can be reversibly oxidized at the bioanode to produce electrons and thus, electricity. When the electrocatalyst is adjacent to the electron conductor, the electrocatalyst and electron conductor are in electrical contact with each other, but not necessarily in physical contact with each other. In one embodiment, the electron conductor is part of, associates with, or is adjacent to an electrocatalyst for an electron mediator.

Generally, the electrocatalyst can be an azine, a conducting polymer or an electroactive polymer. Exemplary electrocatalysts are methylene green, methylene blue, luminol, nitro-fluorenone derivatives, azines, osmium phenanthrolinedione, catechol-pendant terpyridine, toluene blue, cresyl blue, nile blue, neutral red, phenazine derivatives, tionin, azure A, azure B, toluidine blue O, acetophenone, metallophthalocyanines, nile blue A, modified transition metal ligands, 1,10-phenanthroline-5,6-dione, 1,10-phenanthroline-5,6-diol, [Re(phen-dione)(CO)₃Cl], [Re(phen-dione)₃](PF₆)₂, poly(metallophthalocyanine), poly(thionine), quinones, diimines, diaminobenzenes, diaminopyridines, phenothiazine, phenoxazine, toluidine blue, brilliant cresyl blue, 3,4-dihydroxybenzaldehyde, poly(acrylic acid), poly(azure I), poly(nile blue A), poly(methylene green), poly(methylene blue), polyaniline, polypyridine, polypyrole, polythiophene, poly(thieno[3,4-b]thiophene), poly(3-hexylthiophene), poly(3,4-ethylenedioxypyrrole), poly(isothianaphthene), poly(3,4-ethylenedioxythiophene), poly(difluoroacetylene), poly(4-dicyanomethylene-4H-cyclopenta[2,1-b;3,4-b]dithiophene), poly(3-(4-fluorophenyl)thiophene), poly(neutral red), a protein, a metalloprotein, stellacyanin, or combinations thereof. In one preferred embodiment, the electrocatalyst for the electron mediator is poly(methylene green).

4. Enzyme

An enzyme catalyzes the oxidation of the fuel fluid at the bioanode. As enzymes also reduce an oxidant at the biocathode, they are more generally described above at I.A.1.d. Generally, naturally-occurring enzymes, man-made enzymes, artificial enzymes and modified naturally-occurring enzymes can be utilized. In addition, engineered enzymes that have been engineered by natural or directed evolution can be used. Stated another way, an organic or inorganic molecule that mimics an enzyme's properties can be used in an embodiment of the present invention.

Specifically, exemplary enzymes for use in a bioanode are oxidoreductases. In one preferred embodiment, the oxidoreductases act on the CH—OH group or CH—NH group of the fuel (alcohols, ammonia compounds, carbohydrates, aldehydes, ketones, hydrocarbons, fatty acids and the like).

In another preferred embodiment, the enzyme is a dehydrogenase. Exemplary enzymes in this embodiment include alcohol dehydrogenase, aldehyde dehydrogenase, formate dehydrogenase, formaldehyde dehydrogenase, glucose dehydrogenase, glucose oxidase, lactatic dehydrogenase, lactose dehydrogenase, pyruvate dehydrogenase, or lipoxygenase. Preferably, the enzyme is an alcohol dehydrogenase (ADH).

When ethanol is used as a fuel, the enzymes of Krebs cycle can be used. For example, aconitase, fumarase, malate dehydrogenase, succinate dehydrogenase, succinyl-CoA synthetase, isocitrate dehydrogenase, ketoglutarate dehydrogenase, citrate synthase and combinations thereof can be used in the bioanode.

In a presently preferred embodiment, the enzyme is a PQQ-dependent alcohol dehydrogenase. PQQ is the coenzyme of PQQ-dependent ADH and remains electrostatically attached to PQQ-dependent ADH and therefore the enzyme will remain in the membrane leading to an increased lifetime and activity for the biofuel cell. The PQQ-dependent alcohol dehydrogenase enzyme is extracted from gluconobacter. When extracting the PQQ-dependent ADH, it can be in two forms: (1) the PQQ is electrostatically bound to the PQQ-dependent ADH or (2) the PQQ is not electrostatically bound the PQQ-dependent ADH. For the second form where the PQQ is not electrostatically bound to the PQQ-dependent ADH, PQQ is added to the ADH upon assembly of the bioanode. In a presently preferred embodiment, the PQQ-dependent ADH is extracted from gluconobacter with the PQQ electrostatically bound.

5. Enzyme Immobilization Material

As described above, an enzyme immobilization material is utilized in the biofuel cell at the bioanode and/or the biocathode. Further detail regarding the composition of the enzyme immobilization material and the immobilization mechanism can be found above at I.A.5. In one embodiment, the bioanode's enzyme immobilization material is permeable to the fuel fluid and immobilizes and stabilizes the enzyme. The immobilization material is permeable to the fuel fluid so the oxidation of the fuel fluid at the bioanode can be catalyzed by the immobilized enzyme. Preferably, the enzyme immobilization material is a hydrophobically modified polysaccharide, particularly, a hydrophobically modified chitosan or a hydrophobically modified alginate.

6. Bioanode Embodiments

In a further embodiment, the electron mediator can be physically bound to the enzyme. The physical bond can be a covalent or ionic bond between the electron mediator and the enzyme. In still another embodiment, if the electron mediator is capable of reversible electrochemistry at the electron conductor, the electron mediator can be physically bound to the enzyme and the electron mediator can also be physically bound to the electron conductor.

In still another embodiment, the electron mediator is immobilized in the immobilization material. In a preferred embodiment, the electron mediator is oxidized NAD⁺ immobilized in a hydrophobically modified chitosan or a hydrophobically modified alginate membrane. In this embodiment, after the fuel fluid is added to the cell, the NAD⁺ is reduced to NADH and the NADH can diffuse through the hydrophobically modified chitosan membrane or through the hydrophobically modified alginate membrane.

Methods of making and using bioanodes, which are useful in the manufacture and use of biofuel cells comprising the instant biocathode, are known in the art. A preferred bioanode is described in U.S. patent application Ser. No. 10/617,452 (published as U.S. Patent Application Publication No. 2004/0101741), which is incorporated herein by reference in its entirety. Other potentially useful bioanodes are described in U.S. Pat. Nos. 6,531,239 and 6,294,281, which are also incorporated herein by reference.

C. Fuel Fluid and Oxidant

A fuel fluid that can be oxidized to produce electrons at the bioanode and an oxidant that can be reduced to produce water at the biocathode are components of the biofuel cell of this invention.

The fuel fluid for the bioanode is consumed in the oxidation reaction of the electron mediator and the immobilized enzyme. The fuel fluid's molecular size is small enough so the diffusion coefficient through the enzyme immobilization material is large. Exemplary fuel fluids are hydrogen, ammonia, alcohols (such as methanol, ethanol, propanol, isobutanol, butanol and isopropanol), allyl alcohols, aryl alcohols, glycerol, propanediol, mannitol, glucuronate, aldehyde, carbohydrates (such as glucose, glucose-1, D-glucose, L-glucose, glucose-6-phosphate, lactate, lactate-6-phosphate, D-lactate, L-lactate, fructose, galactose-1, galactose, aldose, sorbose and mannose), glycerate, coenzyme A, acetyl Co-A, malate, isocitrate, formaldehyde, acetaldehyde, acetate, citrate, L-gluconate, beta-hydroxysteroid, alpha-hydroxysteroid, lactaldehyde, testosterone, gluconate, fatty acids, lipids, phosphoglycerate, retinal, estradiol, cyclopentanol, hexadecanol, long-chain alcohols, coniferyl-alcohol, cinnamyl-alcohol, formate, long-chain aldehydes, pyruvate, butanal, acyl-CoA, steroids, amino acids, flavin, NADH, NADH₂, NADPH, NADPH₂, hydrocarbons, amines, and combinations thereof. In a preferred embodiment, the fuel fluid is an alcohol, more preferably methanol and/or ethanol; and most preferably ethanol.

The oxidant for the biocathode is consumed in the reduction reaction of the electron mediator and the immobilized enzyme using electrons supplied by the bioanode. The oxidant's molecular size is small enough so the diffusion coefficient through the enzyme immobilization material is large. A variety of means of supplying a source of the oxidant known in the art can be utilized.

In a preferred embodiment, the oxidant is gaseous oxygen, which is transported to the biocathode via diffusion. In another preferred embodiment, the oxidant is a peroxide compound.

The biofuel cells of the embodiments can comprise (i) a bioanode as described above; (ii) a biocathode as described above; (iii) a bioanode and a biocathode as described above; (iv) a bioanode as described above and a biocathode as described in U.S. patent application Ser. No. 10/931,147 (published as U.S. Patent Application Publication No. 2005/0095466); and (v) a bioanode as described in U.S. patent application Ser. No. 10/617,452 (published as U.S. Patent Application Publication No. 2004/0101741) and a biocathode as described above.

The biofuel cell of the instant invention may comprise a polymer electrolyte membrane (“PEM” or salt bridge, e.g., Nafion® 117) to separate the anode compartment from the cathode compartment. However, for embodiments having a bioanode and a biocathode, a PEM is not necessary and a membraneless biofuel cell is produced. The preferential selectivity of the enzymes used in the bioanode and biocathode for catalysis of either the oxidant or the fuel fluid reaction makes the physical separation of the anode compartment from the cathode compartment unnecessary.

### II. Microfluidic Biofuel Cell

Among the various aspects of the invention is a microfluidic biofuel cell utilizing a fuel fluid to produce electricity via enzyme mediated redox reactions taking place at micromolded microelectrodes with immobilized enzymes therein. As in a standard biofuel cell, the bioanode is the site for an oxidation reaction of a fuel fluid with a concurrent release of electrons. The electrons are directed from the bioanode through an electrical connector to some power consuming device. The electrons move through the device to another electrical connector, which transports the electrons to the biofuel cell's biocathode where the electrons are used to reduce an oxidant to produce water. In this manner, the biofuel cell of the present invention acts as an energy source (electricity) for an electrical load external thereto. To facilitate the fuel fluid's redox reactions, the microelectrodes comprise an electron conductor, an electron mediator, an electrocatalyst for the electron mediator, an enzyme, and an enzyme immobilization material.

Unlike a standard biofuel cell, however, the biofuel cell of the invention utilizes at least one micromolded electrode. In one embodiment, the micromolded electrode has a flow through structure that allows fuel to flow within the microelectrode. When compared to conventional biofuel cell electrodes, this structure yields a higher current density because of the higher amount of microelectrode surface area in contact with the fuel. In another embodiment, the micromolded electrode has an irregular topography. Again, the current density of the microelectrode is greater than conventional biofuel cell electrodes because of a higher amount of surface area in contact with the fuel. These features combine with other features disclosed herein to create a biofuel cell with increased current density over conventional biofuel cells from a dimensionally smaller source. Finally, the method of the current invention can advantageously be used to economically produce disposable fuel cells.

A. Microfluidic Channel

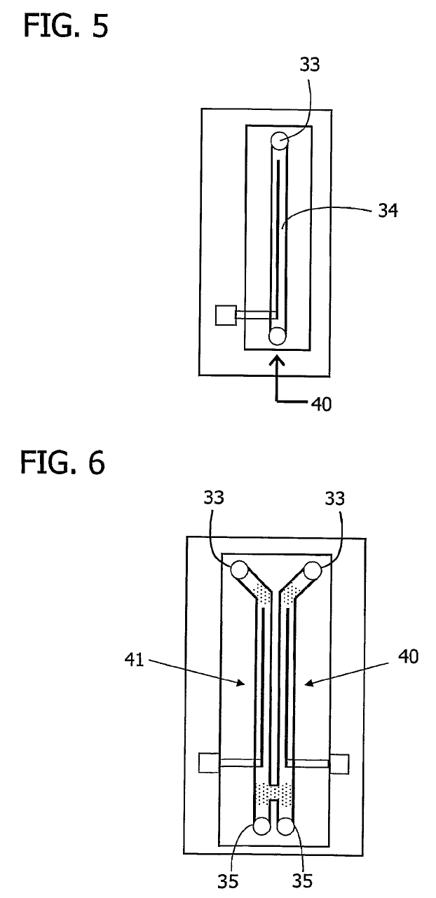

Beyond the bioanode and/or biocathode, the microfluidic biofuel cell is characterized by at least one microfluidic channel that, in service, houses the bioanode and/or the biocathode, the fuel fluid, and the oxidant. The microfluidic channel's configuration can vary depending on the application. In one embodiment, the microfluidic channel can simply be a rectangular chamber with the bioanode and/or the biocathode of the biofuel cell contained therein. See FIG. 5. In other embodiments, the configuration of the microfluidic channel can be more elaborate for any desired purpose, such as to ensure that the bioanode solution and the biocathode solution do not come into physical contact with one another. See FIG. 6.

With reference to FIGS. 5 and 6, the fuel fluid and/or oxidant flow through the microfluidic channel (34), over or through the microelectrode(s), from one end of the microfluidic channel (entry) (33) to the opposite end (exit) (35). In FIG. 6, the bioanode is represented by (41) and the biocathode is represented by (40). The microfluidic channel should facilitate convective flow of the fuel fluid and/or oxidant over the microelectrode(s) while preventing leakage of the same outside the microfluidic channel (34).

B. Electrical Connectors

The electrical connectors provide electrical contact from the microelectrodes to the electrical load external to the microfluidic biofuel cell. In the most general sense, the electrical connector can be any material and structure that facilitates the transfer of electrons from the bioanode to the electrical load and back to the biocathode. In one preferred embodiment, the electrical connector of the microfluidic biofuel cell provide attachment leads to which another device can make physical and electrical contact. This other device, e.g. copper wire, then transports electrons are transported to and from the external electrical load.

In one preferred embodiment, the electrical connector is a thin layer connector that is formed on the microfluidic biofuel cell's substrate prior to other processing. In this embodiment, the subsequently formed microelectrodes are arranged such that they intersect their respective electrical connectors. In an alternative embodiment, the electrical connector is a cylindrical body of electrically conductive material that is attached to the microelectrodes subsequent to their processing.

### III. Microfluidic Biofuel Cell Fabrication

In fabricating a microfluidic biofuel cell in accordance with this invention, a substrate is used on which the other biofuel cell components are constructed. In a preferred embodiment, the first step is to form the electrical connectors, followed by the fabrication of the microelectrodes, and the optional step of defining a biofuel chamber. In an alternative embodiment, the electrical connectors are formed subsequent to the other features.

A. Fabrication of Electrical Connectors

The microfluidic biofuel cell of the invention is formed by providing a substrate onto which the remaining components are formed. The substrate can be made of any material that is not conductive, will not passivate the conductive material of the microelectrode, to which the conductive material will adhere throughout processing, and to which molds can be reversibly sealed. In one embodiment, the substrate is glass. In a preferred embodiment, the substrate is poly(dimethylsiloxane) (PDMS). In another preferred embodiment, the substrate is polycarbonate. In one embodiment, the substrate is flat. In alternative embodiments, the substrate can take on a geometric shape that advantageously suits the particular application.

In a preferred embodiment, the first biofuel cell feature formed on the substrate is an electrical connector, which will be in electrical contact with the microelectrodes in the completed biofuel cell to provide the means for connecting the external electrical load to the microelectrodes. The connector can be made of any electrically conductive material. Exemplary materials include platinum, palladium, gold, alloys of those precious metals, carbon, nickel, copper and stainless steel. In a preferred embodiment, the connector is made of platinum.

The connector can be formed on the substrate using conventional photolithographic techniques known in the silicon wafer industry. For example, to form a thin layer platinum electrical connector, a titanium adhesion layer is first sputtered onto the substrate. This is followed by sputtering a layer of platinum over the titanium layer. Both sputtering processes can be carried out, for example, in an argon-ion sputtering system. The connectors will then be defined by photolithography, with photoresist applied to the platinum layer to protect the desired connector locations. Chemical etching of the two layers with commercially available etchants followed by stripping of the photoresist will yield the finished platinum electrical connectors. In an alternative embodiment, the electrical connectors are the last feature formed. This embodiment is detailed below at III.B.6.

B. Fabrication of Microelectrodes

Following the creation of electrical connectors on the biofuel cell's substrate, the next step is the fabrication of the bioanode and the biocathode. These can be formed in succession or simultaneously.

1. Bioanode Fabrication

In one embodiment, the bioanode and the biocathode are formed on the substrate in succession, where the order of formation is not critical. For the purposes of presentation only, the bioanode fabrication will be detailed first. The first step of fabricating a microscale bioanode is creating a pattern of a microchannel in the surface of a casting mold. In general, the casting mold can be made of any material that is not conductive, will not passivate the conductive material and is able to be reversibly sealed to the substrate, with exemplary materials including silicon, glass, and polymers. The casting mold is preferably made of a polymer, even more preferably made of PDMS. Most preferably, the casting mold is made of polycarbonate.

In a preferred embodiment where the casting mold is a polymer, the pattern is created by using known soft lithography techniques to produce the microchannel in the casting mold to define the shape and size of the bioanode. Soft lithography techniques generally entail the process of molding a prepolymer against a lithographically-defined master that has a raised image of the desired design. The soft lithography technique employed should be able to yield microchannels in the casting mold between about 1 μm to about 1 mm, between about 1 μm to about 200 μm, preferably between about 10 μm to about 200 μm, more preferably between about 10 μm to about 100 μm, and most preferably as small as about 10 μm or less. Exemplary soft lithography techniques include near-field phase shift lithography, replica molding, microtransfer molding (μTM), solvent-assisted microcontact molding (SAMIM), and microcontact printing (μCP). Preferably, the microchannels are formed using replica molding.

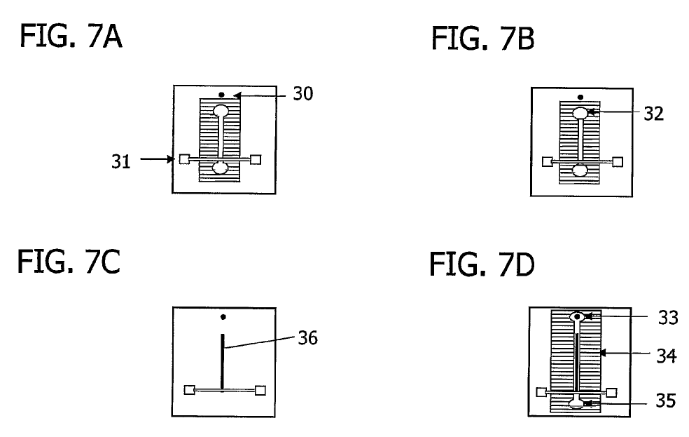

After the microchannel is formed in the casting mold, the patterned side of the casting mold is adhered to the substrate to complete the mold of the microelectrode. See FIG. 7(a). In the embodiment where the electrical connector (31) has previously been formed on the substrate, the microchannel should align over the electrical connector such that the finished microelectrode will be in electrical contact with the connector. Further, a tubing connector (30) is adhered to the substrate to maintain the position that will later become the entry reservoir.

Next, with reference to FIG. 7(b), an electron conductor solution is flowed into the casting mold's microchannel through an entry reservoir (32) that has been created in the casting mold at one end of the microchannel. This entry reservoir (32) is analogous to a pouring basin in the traditional art of metal casting. Excess solution will exit the microchannel at a vent located at the end of the microchannel opposite the entry reservoir.

The electron conductor solution can be any solution that comprises an electron conductor source and a liquid carrier that can be removed via curing to yield a solid microelectrode. The numerous potential electron conductor materials are listed above in I.A.1. In one preferred embodiment, the electron conductor source is a carbon source. In a more preferred embodiment, the electron conductor source is a carbon-based ink. In one such embodiment, the liquid carrier is a carbon-based ink thinner, e.g., Ercon N160 Solvent Thinner. Depending on the nature of the liquid carrier in the solution, two types of microelectrode structures can be formed according to the invention—solid microelectrodes or flow through microelectrodes. With lower viscosity liquid carriers, solid microelectrodes are produced. These microelectrodes are substantially continuous and solid, and fuel fluid flows over such microelectrodes during use. With higher viscosity liquid carriers, flow through microelectrodes are produced with a structure enabling fuel fluid to flow therethrough during use, effectively increasing the surface area of the microelectrode in contact with the fuel fluid.

Regardless of the particular structure, a microelectrode formed in accordance with this invention has several advantages over microelectrodes formed using traditional processes, which necessarily have flat topography. As such, any fluid flowing over conventional microelectrodes has a generally regular flow pattern and is in contact with a generally defined amount of microelectrode surface area. This flat geometric surface area is calculated by adding the rectangular surface area of the top and sides of the flat microelectrode. As current production of a microelectrode is determined in large part by the surface area in contact with the fuel fluid, a flat microelectrode's current production capabilities can only be increased by increasing its size. In contrast, microelectrodes formed in accordance with this invention have highly irregular, three dimensional topography, which yields at least two distinct advantages. First, the effective surface area of the invention's microelectrode is substantially increased compared to a flat screen printed microelectrode. The effective surface area of the microelectrodes herein described is the sum of surface area of the individual peaks and valleys characterizing the microelectrode's topography. One accurate method of calculating this effective surface area is to compare the current output of a microelectrode formed according to the invention with a flat microelectrode of the same length, width, and height dimensions. For example, such analysis of microelectrodes has shown current output of 9.85×10⁻⁴ A/cm² for a microelectrode of this invention, compared to 2.06×10⁻⁴ A/cm² for a conventional glassy carbon electrode. Further, the microelectrode's irregular topography can create turbulent flow of the fluid. Such a flow pattern is advantageous because it induces mixing of the fluid over the microelectrode, which in turn increases the transport rate of the fluid to the microelectrode. Increasing the transport rate of the fluid facilitates the reactions taking place within the microelectrode, thereby increasing the microelectrode's current load capability.

In one alternative embodiment, a primer is flowed into the casting mold's microchannels and quickly dried prior to introducing the electron conductor solution. The primer can be any material that will help prevent the electron conductor from becoming semi-permanently attached to the casting mold. For example, in the carbon-based ink embodiment, carbon-based ink thinner can be used as a primer, if one is desired.

After the solution fills the casting mold's microchannels, heat is applied to cure the electron conductor solution. In general, heating should be conducted at a temperature sufficient to remove the liquid carrier from the solution, but low enough so that the resulting microelectrode is not damaged. In one preferred embodiment, heating occurs at about 75□C. Also, heat should be applied for a time sufficient to remove substantially all of the liquid carrier from the solution. In one preferred embodiment, heat is applied for at least about one hour. In another preferred embodiment, heating occurs at about 75° C. for about one hour. With reference to FIG. 7(c), the curing process yields a solidified microelectrode (36) that is approximately 20% smaller than the original size of the casting mold's microchannel(s) due to evaporation of the carrier.

In the method according to the invention, the microelectrode is treated to impart an electron mediator, an optional electrocatalyst for the electron mediator, an enzyme, and an enzyme immobilization material thereto to form a bioanode via one of at least four embodiments. In a first embodiment, the enzyme immobilization material containing the enzyme is applied to the cured microelectrode, followed by the introduction of the electron mediator and the optional electrocatalyst. To form the bioanode, the casting mold is removed from the substrate after curing the microelectrode. See FIG. 7(c). With reference to FIG. 7(d), in place of the casting mold, a gas-permeable mold with a microchannel (34) approximately twice the width of the casting mold's microchannel is reversibly sealed over the microelectrode. The gas-permeable mold can be made of any material that is not conductive, will not passivate the electron conductor and facilitates evaporation of a solvent. Preferably, a silicon polymer, such as PDMS, is used as the gas-permeable mold material. More preferably, a thermoplastic resin, such as polycarbonate, is the gas-permeable mold material. After the gas-permeable mold is in place, an enzyme immobilization material containing a bioanode enzyme is applied to the cured microelectrode. This is accomplished by syringe pumping the casting solution into the entry reservoir (33) and through the gas-permeable mold to an exit vent (35). At this point, an electron mediator solution optionally comprising an electrocatalyst is hydrodynamically flowed through the gas-permeable mold's microchannel using an entry reservoir (33) and a vent (35) as described above. With the width of the microchannel approximately twice the width of the microelectrode, a small amount of the electron mediator solution will inevitably coat onto the substrate; however, this ensures that the entire microelectrode is properly coated. The electron mediator solution's solvent is then allowed to evaporate through the gas-permeable mold or through an entry reservoir and/or vent in the mold, leaving a bioanode. If the electron mediator needs to be polymerized, an electropolymerization process can be utilized to that end. This embodiment is less desirable if the electron mediator needs to be electropolymerized. See FIG. 7(d) for a finished bioanode.

Therefore, in a more preferred second embodiment, the electron mediator and the optional electrocatalyst are applied to the solidified microelectrode, the electron mediator is electropolymerized if needed, and then the enzyme immobilization material containing the enzyme is applied to the microelectrode. In the second embodiment, the casting mold is removed from the substrate after curing the microelectrode. In place of the casting mold, a gas-permeable mold as detailed above is reversibly sealed over the microelectrode. Here, an electron mediator solution optionally comprising an electrocatalyst is hydrodynamically flowed through the gas-permeable mold's microchannel using an entry reservoir and a vent as described above. Again, a small amount of the electron mediator solution will inevitably coat onto the substrate, but this ensures that the entire microelectrode is properly coated. The electron mediator solution's solvent is then allowed to evaporate through the gas-permeable mold, leaving an electron mediator coated microelectrode. If the electron mediator needs to be polymerized, an electropolymerization process can be utilized to that end. Next, an enzyme immobilization material containing a bioanode enzyme is applied to from the bioanode. This is accomplished by syringe pumping a solution containing the enzyme immobilization material and the bioanode enzyme into the entry reservoir and through the gas-permeable mold.

In an even more preferred third embodiment, the electron mediator and the optional electrocatalyst are introduced to the electron conductor solution prior to injection into the casting mold, and after curing, the enzyme immobilization material containing the enzyme is applied to the cured microelectrode. In the third embodiment, the electron mediator and the optional electrocatalyst are suspended in the electron conductor solution prior to introduction into the casting mold's microchannel. The modified electron conductor solution is then flowed into the casting mold's microchannel and cured, as detailed above at III.A. This embodiment advantageously enhances the bioanode's conductivity, increases simplicity by eliminating a processing step, and improves electron mediator transport efficiency. The embodiment also yields a highly conductive composite bioanode with the selectivity properties of the individual electron mediator, while also possessing the transport efficiency of a gas diffusion style anode. Electropolymerization of the electron mediator can be carried out at this time if required. Thereafter, an enzyme immobilization material containing a bioanode enzyme is applied to the modified microelectrode to form the bioanode. This is accomplished by syringe pumping a solution containing the enzyme immobilization material and the bioanode enzyme into the entry reservoir and through the gas-permeable mold.

In the most preferred fourth embodiment, the electron mediator, the optional electrocatalyst, and the enzyme immobilization material containing the enzyme are all combined in the electron conductor solution prior to injection into the casting mold to produce, upon curing, a complete bioanode according to the invention. In the fourth and most preferred embodiment, the electron mediator, the optional electrocatalyst, and the enzyme immobilization material containing the enzyme are all combined in the electron conductor solution. The solution is then introduced into the casting mold as detailed above. Curing the modified solution forms a complete bioanode according to the invention. This embodiment represents the simplest bioanode formation technique, eliminating excess steps and molds required by the other embodiments.

In all embodiments, the specific composition of the enzyme immobilization material, the enzyme, the electron mediator, and the optional electrocatalyst is detailed above in I.B.2.-I.B.4. The preferred enzyme immobilization material for the bioanode is a hydrophobically modified polysaccharide, particularly, a hydrophobically modified chitosan or a hydrophobically modified alginate. The preferred enzyme at the anode is an alcohol dehydrogenase. When an electron mediator/electrocatalyst combination is employed, they are preferably NAD⁺ and poly(methylene green) respectively. If an electron mediator that provides reversible electrochemistry is used, the preferred electron mediator is PQQ. Also, the casting mold can include more than one microchannel in all embodiments.

2. Biocathode Fabrication

To form a biocathode in accordance with the invention, the same general processing steps taken to fabricate the bioanode can be used to produce a biocathode. The four general embodiments for treating the biocathode with the enzyme immobilization material, the enzyme, the electron mediator, and the electrocatalyst are the same as those for the bioanode, though the option of omitting the electrocatalyst is not applicable. The specific composition of the enzyme immobilization material, the enzyme, the electron mediator, and the electrocatalyst is detailed above in I.A.2.-I.A.5. The preferred enzyme immobilization material for the biocathode is a hydrophobically modified polysaccharide, particularly, a hydrophobically modified chitosan or a hydrophobically modified alginate. Additionally for the cathode, the preferred enzyme is bilirubin oxidase, the preferred electron mediator is bilirubin, and the preferred electrocatalyst is Ru(bpy)₃²⁺ in a modified membrane.

C. Forming the Operational Biofuel Cell

After the bioanode and biocathode have been formed in accordance with this invention, the casting or gas-permeable molds are optionally removed. In this optional embodiment the bioanode and biocathode remain on the substrate. After the casting or gas-permeable molds are removed, a microfluidic channel form is aligned over the bioanode and biocathode. This form is micropatterned so as to create at least one microfluidic channel through which the biofuel cell's fuel fluid can flow. The form can be made of any material that is not conductive, will not passivate the conductive material and will adhere to the substrate. Preferably, the form is PDMS. More preferably, this overlay is polycarbonate. The micropatterns of the microfluidic channel(s) in the form can be created by using any known soft lithography technique. In one embodiment, the microfluidic channel is about two to four times larger than the microelectrodes. In another embodiment, the microfluidic channel is approximately the same size as the microelectrodes. The microfluidic channels of the form essentially define the electrochemical cell in which the fuel fluid will interface with the microelectrodes. When only one microfluidic channel is used to house the bioanode, biocathode, fuel fluid, and oxidant, the mixture of fuel fluid and oxidant in the same microfluidic chamber does not compromise the function of the microelectrodes of the invention because their redox reactions are selective. Stated another way, the bioanode will only react with fuel fluid and the biocathode will only react with the oxidant, and no cross reaction takes place.

In an alternative embodiment, the casting or gas-permeable mold(s) remain in contact with the substrate and serves to define the microfluidic channels of the biofuel cell, acting as the microfluidic channel form described above. In this embodiment, the fuel fluid travels through the space between the microchannels of the mold(s) and the bioanode or biocathode. In this embodiment, subsequent processing must be performed to create a junction between the individual bioanode and biocathode microfluidic channels. To form the junction, a passage connecting the individual microfluidic chambers is formed in the mold(s) by any appropriate means, such as applying a perpendicular force to the top of the mold(s) or removing sufficient material from the mold(s). Thereafter, the passage is covered by a material that will seal the junction to inhibit leakage of the fuel fluid or oxidant during operation. The material must be capable of being joined to the mold material to create the appropriate seal. In one embodiment, the covering material is simply a flat piece of the mold material, such as PDMS or polycarbonate.

D. Optional Formation Embodiments

The microelectrode fabrication technique described above in III.B.1. refers to the embodiment wherein the bioanode and the biocathode were formed successively, which was followed by a method of connecting the bioanode and biocathode via microchannels to form the biofuel cell. In an alternative embodiment, the bioanode and the biocathode can be formed simultaneously. In this embodiment, a single casting mold is patterned to form both the bioanode and biocathode. Alternatively, a combination of casting molds can be used to form the individual bioanode and biocathode. In either case, after the bioanode and biocathode are simultaneously formed, the operational biofuel cell is formed by either applying a microfluidic channel form or modifying the casting mold(s) as detailed above in III.B.3.

The embodiment described above in III.A describes the formation of the electrical connectors on the substrate prior to other processing steps. In an alternative embodiment, the electrical connectors are added to the microfluidic biofuel cell as a final processing step. Here, holes are created in the microfluidic channel form or the modified casting mold(s) to expose a portion of each bioanode and biocathode. Next, electrical connectors are physically joined to the exposed portion of each bioanode and biocathode. In this embodiment, the electrical connectors can be any material in any structure that will enable the external electrical load to make electrical contact with the bioanode and biocathode. In one preferred embodiment, the electrical connectors are cylindrical copper bodies. Further, any joining technique capable of maintaining the electrical contact between the electrical connectors and the bioanode and biocathode can be employed. In one preferred embodiment, silver epoxy paste can be used to join the electrical connectors and the bioanode and biocathode electrically. This embodiment has the advantage of increasing the conductivity between these components.

The above embodiments have described a biofuel cell wherein both the bioanode and the biocathode are housed within the microchannel(s) of the biofuel cell. While this is the preferred embodiment, alternative embodiments of the invention include an anode or a cathode located external to the microchannel(s) of the biofuel cell. Here, a fuel cell is formed by combining a microfluidic bioanode or biocathode with the appropriate external anode or cathode.

E. Use of the Microfluidic Biofuel Cell

After fabrication of the operational microfluidic biofuel cell of this invention is complete, it can be utilized in myriad applications where a fluid fuel source and oxidant are available for the bioanode and biocathode respectively. In use, the fuel fluid and the oxidant travel through the microfluidic channel(s) to contact the bioanode and biocathode. There, the redox reactions described above at I. take place to create a current source. The microfluidic biofuel cell of the instant invention may be used in any application that requires an electrical supply, such as electronic devices, commercial toys, internal medical devices, and electrically powered vehicles. Further, the microfluidic biofuel cell of the instant invention may be implanted into a living organism, wherein the fuel fluid is derived from the organism and current is used to power a device implanted in the living organism.

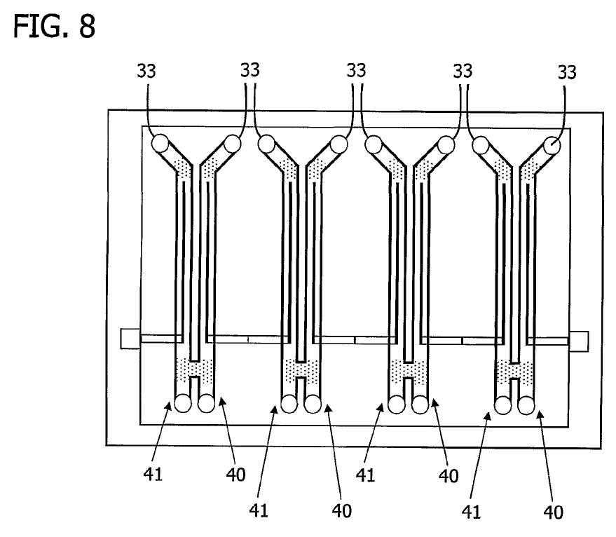

In addition, multiple microfluidic biofuel cells of the invention can be joined in a series electrical circuit to form a biofuel cell stack. See FIG. 8. A series stack is formed by electrically joining the bioanode (41) of one biofuel cell to the biocathode (40) of another biofuel cell, which is in turn connected to another bioanode (41) until the desired stack is obtained. Fuel fluid and/or oxidant flows into the microfluidic chamber in an entry reservoir (33). By forming stacks, the total voltage output of a microfluidic biofuel cell circuit is theoretically the sum of the voltage output from the individual microfluidic biofuel cells in series. The greater overall voltage output of such a stack is useful in supplying electricity to electronic devices, toys, medical devices, and vehicles with power requirements higher than an individual microfluidic biofuel cell could provide.

### IV. Methods of Generating Electricity

The invention includes a method of generating electricity comprising (a) oxidizing the fuel fluid at the anode and reducing the oxidant at the cathode; (b) oxidizing the reduced form of the electron mediator during the reduction of the oxidant at the biocathode; (c) oxidizing the electrocatalyst; and (d) reducing the electrocatalyst at the electron conductor, wherein the electricity is generated using a biofuel cell comprising the bioanodes and/or biocathodes as described above. Another method of generating electricity comprises (a) oxidizing the fuel fluid at the anode and reducing the oxidant at the cathode; (b) oxidizing the reduced form of the electron mediator during the reduction of the oxidant at the biocathode; and (c) reducing the electron mediator at the electron conductor, wherein the electricity is generated using a biofuel cell comprising the bioanodes and/or biocathodes as described above.

### DEFINITIONS

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The term “acyl,” as used herein alone or as part of another group, denotes the moiety formed by removal of the hydroxyl group from the group—COOH of an organic carboxylic acid, e.g., RC(O)—, wherein R is R₁, R₁O—, R₁R₂N—, or R₁S—, R₁ is hydrocarbyl, heterosubstituted hydrocarbyl, or heterocyclo, and R₂ is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

The term “heteroatom” shall mean atoms other than carbon and hydrogen. The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The following examples illustrate the invention.

### EXAMPLES

### Example 1

### Preparation of Alkyl Modified Chitosan

Medium molecular weight chitosan (available from Aldrich) (0.500 g) was dissolved by rapid stirring in 15 mL of 1% acetic acid. This resulted in a viscous gel-like solution and then 15 mL of methanol was added. The chitosan gel was allowed to stir for approximately 15 minutes, then 20 mL aldehyde (butanal, hexanal, octanal, or decanal) was added to the chitosan gel, followed by 1.25 g of sodium cyanoborohydride. The gel was continuously stirred until the suspension cooled to room temperature. The resulting product was separated by vacuum filtration and washed with 150 mL increments of methanol three times. The modified chitosan was then dried in a vacuum oven at 40° C. for two hours, leaving a flaky white solid. One percent by weight suspensions of each of the polymers were formed in 50% acetic acid, chloroform, and t-amyl alcohol.

### Example 2

### Preparation of Ru(bipyridine)₂(4-methyl-4′-(6-bromohexyl)-2,2′-bipyridine)⁺² Modified Chitosan

The preparation of Ru(bipyridine)₂(4-methyl-4′-(6-bromohexyl)-2,2′-bipyridine)⁺² modified chitosan started with the synthesis of a substituted bipyridine, 4-methyl-4′-(6-bromohexyl)-2,2′-bipyridine. To prepare the substituted bipyridine, 50 mL THF containing 1.69 g 4,4′-dimethyl-2,2′-bipyridine was added dropwise over 30 minutes to 4.1 mL of THF containing 9.1 mmol lithium diisopropylamine. This mixture was stirred for 1.5 hours, then cooled to 0° C., followed by dropwise addition of 9.2 mmol dibromoalkane of desired chain length with stirring. This mixture was stirred for 1.5 hours, quenched with ice water, and extracted with ether. The residue was recrystallized 3 times from ethyl acetate. Once the 4-methyl-4′-(6-bromohexyl)-2,2′-bipyridine was prepared, it was reacted to form the Ru(bipyridine)₂(4-methyl-4′-(6-bromohexyl)-2,2′-bipyridine)⁺² by refluxing 1.315 g of Ru(bpy)₂Cl₂ (in its hydrate form), 0.8201 g of 4-methyl-4′-(6-bromohexyl)-2,2′-bipyridine, and 0.76 g sodium bicarbonate in 60 mL of 2:3 methanol-water solution until the Ru(bpy)₂Cl₂ was depleted. The depletion of Ru(bpy)₂Cl₂ was determined by UV-Vis absorption data. The resulting complex was precipitated by adding 4 mL of 3 M ammonium hexafluorophosphate (or a sodium or potassium perchlorate salt), followed by recrystallization from acetone/CH₂Cl₂. This reaction sequence yielded 77% Ru(bipyridine)₂(4-methyl-4′-(6-bromohexyl)-2,2′-bipyridine)⁺².

After its preparation, 137 mg Ru(bipyridine)₂(4-methyl-4′-(6-bromohexyl)-2,2′-bipyridine)⁺² was dissolved in a mixture of 5 mg of deacetylated chitosan in 1% acetic acid and DMF (1:1, 1 mL). This mixture was heated at 90° C. for 12 hours. After the reaction period, acetonitrile was added to precipitate Ru(bipyridine)₂(4-methyl-4′-(6-hexyl)-2,2′-bipyridine)⁺² modified chitosan. The precipitate was collected and purified by dissolution in 1% acetic acid, then recrystallized in methanol and dried under reduced pressure.

### Example 3

### Fluorescence Imaging of Hydrophobically Modified Chitosans

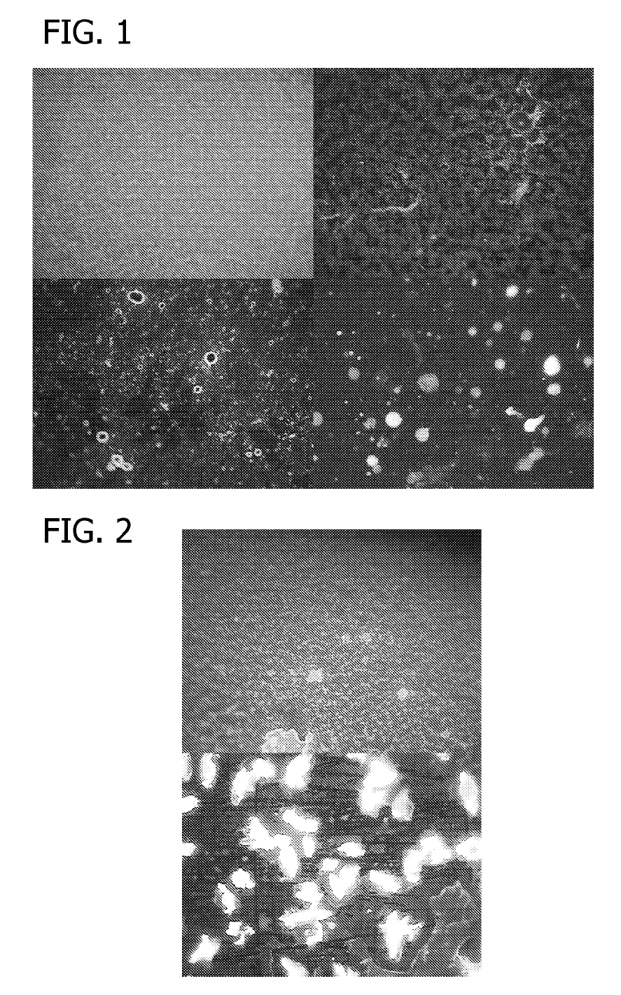

Two microliters of each polymer suspension were cast onto a glass microscope slide (Fisher) and dried in the desiccator. A 20 μL volume of 0.01 mM Ru(bpy)₃²⁺, or 0.01 mM FITC was pipetted onto the polymer cast and allowed to soak for two minutes. After soaking, the slides were rinsed with 18 MΩ water and allowed to dry in the desiccator. The polymers were imaged using an Olympus BX60M epifluorescence microscope (Melville, N.Y.). The polymers were observed under a 40× ultra-long working distance lens with a video camera (Sony SSC-DC50A). Fluorescence excitation was achieved with a mercury lamp. A frame grabber card (Integral Technologies, Inc., Indianapolis, Ind.) was used to acquire images, and the images were analyzed using SPOT software (Diagnostic Instruments, Inc.) on a Dell PC. Fluorescence imaging of each of the hydrophobically modified polyelectrolytes in Ru(bpy)₃⁺² and fluorescein was performed to determine the morphological effects of the hydrophobic modification. FIG. 1 are representative fluorescence micrographs of hydrophobically modified chitosan in Ru(bpy)₃⁺². It can be seen that aggregates form within the hydrophobically modified chitosans and that the morphology changes with alkyl chain length. The butyl modified chitosan appears to have small, fibrous interconnects, whereas the hexyl modified chitosan has large domains containing smaller micellar domains. As the alkyl chain length increases, the number of micellar domains decreases, but the size of the domain increases. Fluorescence micrographs of unmodified chitosan do not show distinct domains, so micellar structure was not observed for unmodified chitosan. FIG. 2 are representative fluorescence micrographs of hydrophobically modified chitosan membranes soaked in FITC. The same morphological changes can be observed with either the cationic or the anionic fluorescent dye.

### Example 4

### Electrochemical Measurements of Hydrophobically Modified Chitosans

Glassy carbon working electrodes (3 mm in diameter, CH Instruments) were polished on a Buehler polishing cloth with 0.05 micron alumina and rinsed in 18 MΩ water. Two microliters of each polymer suspension was cast onto a glassy carbon electrode surface and allowed to dry in a vacuum desiccator until use. Cyclic voltammetry was used to measure the flux of the redox species through the polymer membrane at the electrode surface. The working electrodes were allowed to equilibrate in a 1.0 mM redox species solution containing 0.1 M sodium sulfate as the supporting electrolyte along with a platinum mesh counter electrode and measured against a saturated calomel reference electrode. The redox species studied were caffeine, potassium ferricyanide, and Ru(bpy)₃²⁺. The data were collected and analyzed on a Dell computer interfaced to a CH Instruments potentiostat model 810. Cyclic voltammetry was performed at scan rates ranging from 0.05 V/s to 0.20 V/s. All experiments were performed in triplicate and reported uncertainties correspond to one standard deviation.

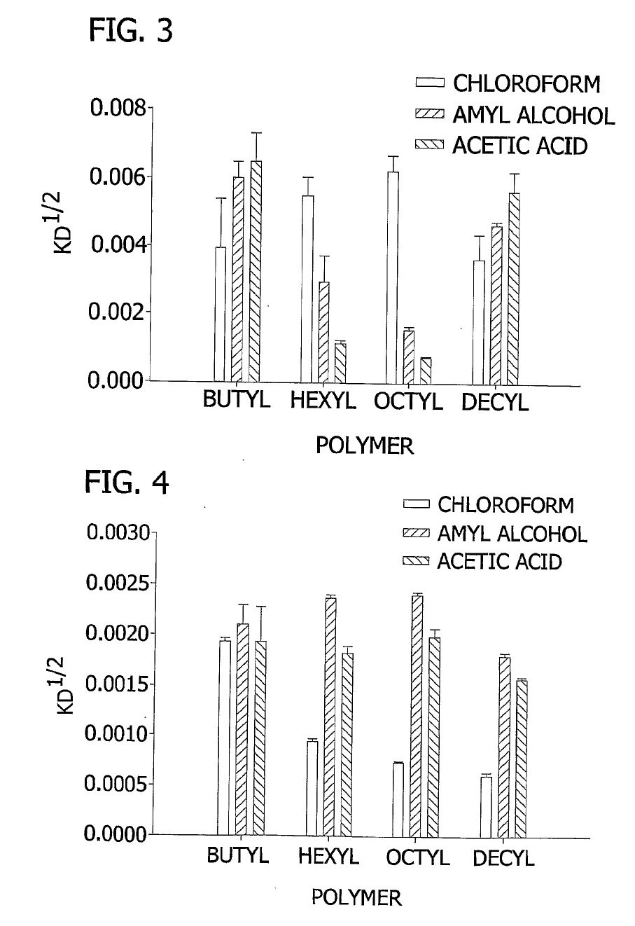

Cyclic voltammetric studies of the two hydrophobically modified polyelectrolytes were conducted as a function of the alkyl chain length of the hydrophobic modification. All cyclic voltammetric experiments showed linear iₚ vs v^{1/2} plots, signifying transport-limited electrochemistry. Since electrochemical flux is a function of concentration as shown in Equation 2, KD^{1/2} values are reported in this paper as a concentration independent method of comparing fluxes.

$$Flux = \frac{i}{nFA} = \frac{2.69 \times {10}^{5} {n}^{3 / 2} AC * {v}^{1 / 2} {KD}^{1 / 2}}{nFA} \qquad Equation 2$$

where i is the peak current, n is the number of electrons transferred, F is Faraday's constant, A is the area of the electrode, C* is the concentration of redox species, v is the scan rate, K is the extraction coefficient, and D is the diffusion coefficient. FIG. 3 shows the KD^{1/2} values for flux of caffeine through hydrophobically modified chitosan as a function of the alkyl chain length of the modifier and the solvent in which the polymer is resuspended. The solvent determines the degree of swelling of the polymer during re-casting. Most literature studies on chitosan and chitosan derivatives employ acetic acid as the solvent for resuspension, however, it is important to note from the KD^{1/2} values for chloroform provides a higher average flux. Unmodified chitosan is only soluble in the acetic acid solution. The KD^{1/2} value for unmodified chitosan in caffeine is 5.52 (±0.14)×10⁻³. It is clear that hydrophobic modification of chitosan can decrease the flux of caffeine, but cannot make appreciable increases in flux.

On the other hand, transport of large, hydrophobic ions, like Ru(bpy)₃⁺², can be greatly affected by small changes in pore structure/size. FIG. 4 shows KD¹¹² values for transport of Ru(bpy)₃⁺² through hydrophobically modified chitosan membranes. The KD^{1/2} value for Ru(bpy)₃⁺² transport through unmodified chitosan is 2.17 (0.33)×10⁻⁴. It is evident that hydrophobic modification of chitosan increases the transport of Ru(bpy)₃⁺² in all cases, by as much as 11.1 fold for octyl modified chitosan membrane resuspended in t-amyl alcohol.

### Example 5

### Preparation of Electrodes

A solution of 2 wt. % of a hydrophobically modified chitosan polymer was suspended in t-amyl alcohol and a solution of glucose oxidase was added. This solution was pipeted onto an electrode material. This electrode material was typically a carbon cloth, or other carbon material.

### Example 6

### Glucose Oxidase Activity Tests for Hydrophobically Modified Chitosans

Glucose oxidase (GOx) catalyzes the oxidation of β-D-glucose to D-glucono-δ-lactone with the concurrent release of hydrogen peroxide. It is highly specific for β-D-glucose and does not act on α-D-glucose. In the presence of peroxidase, hydrogen peroxide enters into a second reaction in the assay involving p-hydroxybenzoic acid and 4-amino antipyrine with the quantitative formation of quinoneimine dye complex, which is measured at 510 nm. The activity of GOx enzyme was measured in each of the hydrophobically modified Nafion and chitosan membranes. The absorbance was measured at 510 nm against water after immobilizing the GOx enzyme within the hydrophobically modified chitosan membranes, and casting it in a plastic vial. All experiments were performed in triplicate and reported uncertainties correspond to one standard deviation.

As described above and tabulated in Table 2, the highest enzyme activity was observed for glucose oxidase in a hexyl modified chitosan suspended in t-amyl alcohol. These immobilization membranes showed a 2.53 fold increase in GOx enzyme activity over enzyme in buffer.

### Example 7

### Chitosan-Butyl Bioanodes

Glucose dehydrogenase. Anodes were made from 1 cm² AvCarb™ carbon paper. The anodes were electropolymerized in 0.4 mM methylene green, 0-1 M sodium nitrate and 10 mM sodium borate by performing cyclic voltammetry from −0.3 V to 1.3 V for 12 sweep segments at a scan rate of 0.05 V/s. They were then rinsed and allowed to completely dry in a vacuum dessicator. Chitosan mixtures were prepared by mixing 0.01 g hydrophobically modified chitosan (butyl, hexyl, octyl or decyl) with 1 mL Nafion® DE 520 and vortexing with mixing beads for 1 hour. A 40 μL aliquot of the chitosan/Nafion® mixture was then mixed with a 20 μL aliquot of glucose dehydrogenase (1 mg enzyme in 10 mL pH 7.15 phosphate buffer) for 1 minute. The chitosan/enzyme mixture was pipetted onto the anode and allowed to completely dry in the vacuum dessicator.

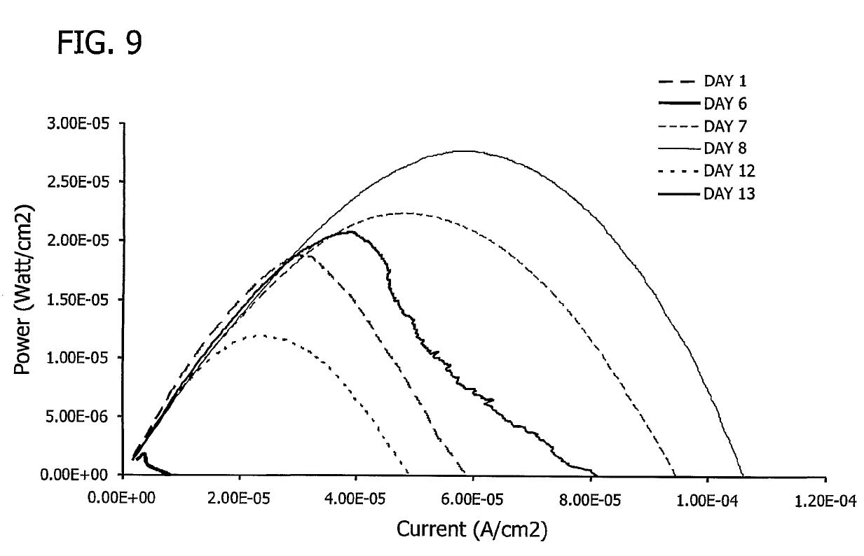

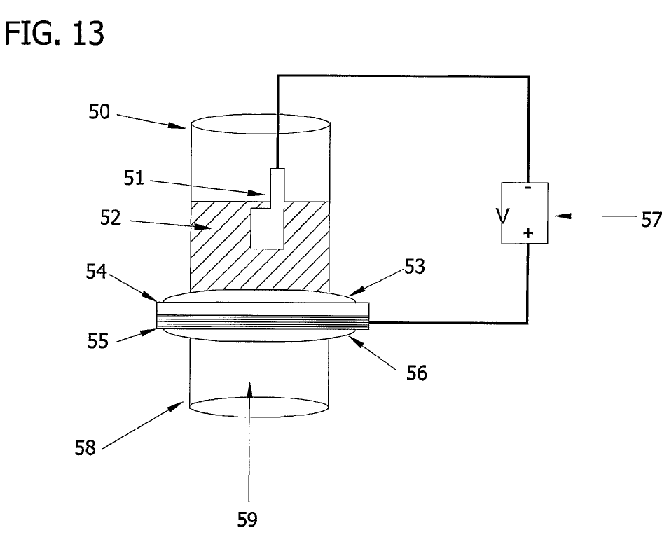

An I-cell setup (FIG. 13) was used so that the fuel cell would be anode dependent and the platinum cathode would not be poisoned from being submerged in buffer solution. An I-cell allows the platinum electrode to operate in air breathing mode. FIG. 13 is a schematic of the I-cell that was used in this experiment. In FIG. 13, a glass tube 50 contains the fuel solution 52 and the bioanode 51 that is immersed in the fuel solution. The glass tube 50 is connected by O-ring 53 to a Nafion® polyelectrolyte membrane 54 and the fuel solution 52 also contacts the Nafion® polyelectrolyte membrane 54. The Nafion® polyelectrolyte membrane 54 is in contact with a 20% platinum gas diffusion electrode cathode 55 that is connected to another glass tube 58 using and O-ring 56. Air 59 can contact the 20% platinum gas diffusion electrode cathode 55 and there is an electrical connection from the cathode 55 to the bioanode 51 through a potentiostat 57. Initially, the fuel used was 1 mM glucose with 1 mM NAD⁺, but after the first week, the fuel concentration was increased to 100 mM glucose with 1 mM NAD⁺. Power curves for a butyl-chitosan bioanode (FIG. 9) were obtained by first allowing the fuel cell to equilibrate and reach an open circuit potential.

Alcohol dehydrogenase. The bioanode containing alcohol dehydrogenase was prepared by the same procedure as described for glucose dehydrogenase above except alcohol dehydrogenase was substituted for glucose dehydrogenase. A biofuel cell in a single compartment cell having a butyl-chitosan bioanode, a platinum cathode, and 1 mM ethanol fuel solution at pH 8 and room temperature and humidity was the subject of a lifetime study. The platinum cathode was coated in a polymer electrolyte membrane. The data from the study is presented in the following table.

| | | Open Circuit | Current | Power |
|---|---|---|---|---|
| Lifetime | Temperature (° C.)/ | Potential | Density | Density |
| (days) | Humidity (%) | (V) | (A) | (W) |
| 1 | 22/55 | 0.6628 | 4.60*10⁻³ | 2.93*10⁻³ |
| 2 | 21/55 | 0.6585 | 5.47*10⁻³ | 3.06*10⁻³ |
| 3 | 21/54 | 0.6940 | 4.20*10⁻³ | 2.50*10⁻³ |
| 4 | 22/64 | 0.7054 | 4.48*10⁻³ | 2.72*10⁻³ |
| 5 | 32/54 | 0.6143 | 5.35*10⁻³ | 2.75*10⁻³ |
| 10 | 21/58 | 0.6675 | 6.00*10⁻³ | 3.41*10⁻³ |
| 15 | 22/56 | 0.6554 | 5.75*10⁻³ | 3.20*10⁻³ |
| 20 | 20/53 | 0.7252 | 6.456*10⁻³ | 4.04*10⁻³ |
| 25 | 21/57 | 0.6780 | 1.019*10⁻² | 5.89*10⁻³ |
| 35 | 21/55 | 0.6542 | 6.32*10⁻³ | 3.45*10⁻³ |
| 45 | 19/50 | 0.6450 | 5.00*10⁻³ | 2.23*10⁻³ |
| 50 | 20/53 | 0.4775 | 1.434*10⁻³ | 8.21*10⁻⁴ |
| 55 | 20/40 | 0.6282 | 8.680*10⁻⁴ | 4.58*10⁻⁴ |

### Example 8

### Chitosan-Butyl Biocathodes

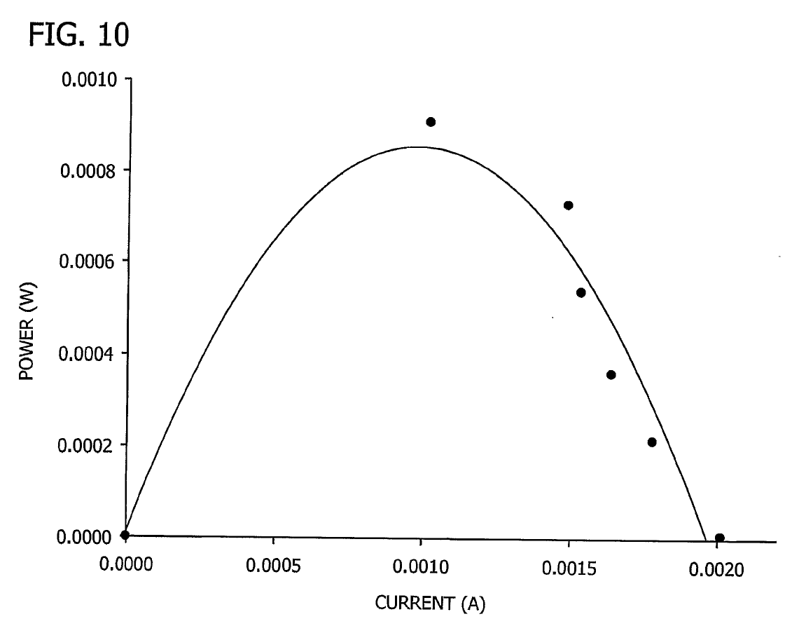

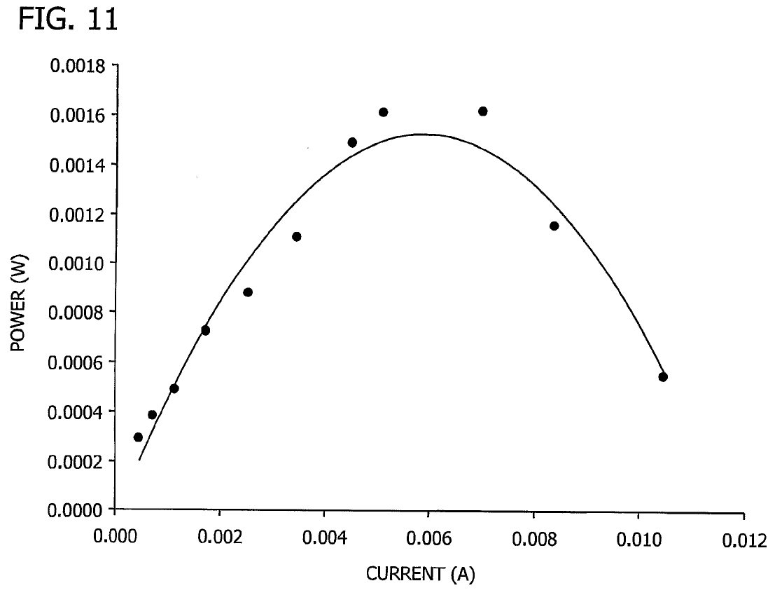

Bilirubin Oxidase. Chitosan mixtures were prepared by mixing 0.01 g hydrophobically modified chitosan (butyl, hexyl, octyl or decyl) with 1 mL Nafion® DE 520 and vortexing with mixing beads for 1 hour. A 40 μL aliquot of the chitosan/Nafion® mixture was then mixed with a 20 μL aliquot of bilirubin oxidase (1 mg enzyme in 10 mL pH 7.15 phosphate buffer) for 1 minute. The chitosan/enzyme mixture was pipetted onto a 1 cm² piece of carbon paper to fabricate the cathode and it was allowed to completely dry in the vacuum dessicator. Data for power curves were collected for a butyl-chitosan bilirubin oxidase cathode combined with either (1) a TBA-modified Nafion® NAD⁺-dependent alcohol dehydrogenase anode (FIG. 10) or (2) butyl-chitosan NAD⁺-dependent alcohol dehydrogenase anode (FIG. 11).

Also, a study to determine the optimum temperature for operation of various biofuel cells was undertaken. The maximum open circuit potential (V), maximum current density (mA)cm²) and maximum power density (mW/cm²) for (1) a TBA-modified Nafion® NAD⁺-dependent alcohol dehydrogenase anode and a butyl-chitosan bilirubin oxidase cathode, (2) a butyl-chitosan NAD⁺-dependent alcohol dehydrogenase anode and a TBA-modified Nafion® bilirubin oxidase cathode, and (3) a butyl-chitosan NAD⁺-dependent alcohol dehydrogenase anode and a butyl-chitosan bilirubin oxidase cathode were measured at various temperatures. This temperature data is presented in the following tables.

TABLE
NAD⁺-dependent TBAB-modified Nafion ® anode
and a butyl-chitosan bilirubin oxidase cathode

| | Results | | |
|---|---|---|---|
| | Maximum Open | Maximum Current | Maximum Power |
| Temperature | Circuit Potential | Density | Density |
| (° C.) | (V) | (mA/cm²) | (mW/cm²) |
| 20 | 1.113 | 8.27e−4 | 8.38e−4 |
| 25 | 1.118 | 1.24e−3 | 1.26e−3 |
| 30 | 1.126 | 1.29e−3 | 1.33e−3 |
| 35 | 1.092 | 6.90e−4 | 6.85e−4 |
| 40 | 1.090 | 9.45e−4 | 9.35e−4 |
| 50 | 1.093 | 1.38e−3 | 1.38e−3 |
| 60 | 1.070 | 1.22e−3 | 1.19e−3 |
| 70 | 0.558 | 3.11e−4 | 1.43e−4 |
| 80 | 0.347 | 9.46e−5 | 2.34e−5 |
| 90 | 0.122 | 2.43e−5 | 5.34e−7 |

TABLE
Butyl-chitosan anode and a TBAB-modified Nafion ® bilirubin
oxidase cathode

| | Results | | |
|---|---|---|---|
| | Maximum Open | Maximum Current | Maximum Power |
| Temperature | Circuit Potential | Density | Density |
| (° C.) | (V) | (mA/cm²) | (mW/cm²) |
| 20 | 0.8078 | 2.69e−4 | 1.90e−4 |
| 25 | 0.8648 | 5.00e−4 | 3.82e−4 |
| 30 | 0.8809 | 6.00e−4 | 4.68e−4 |
| 35 | 0.8896 | 6.54e−4 | 5.76e−4 |
| 40 | 0.8880 | 7.43e−4 | 5.86e−4 |
| 50 | 0.8999 | 9.81e−4 | 7.85e−4 |
| 60 | 0.9100 | 1.021e−4 | 8.27e−4 |
| 70 | 0.804 | 3.80e−4 | 2.66e−4 |
| 80 | 0.489 | 1.81e−4 | 6.78e−5 |
| 90 | 0.1963 | 7.23e−5 | 6.93e−6 |

TABLE
Butyl-chitosan anode and a butyl-chitosan
bilirubin oxidase cathode

| | Results | | |
|---|---|---|---|
| | Maximum Open | Maximum Current | Maximum Power |
| Temperature | Circuit Potential | Density | Density |
| (° C.) | (V) | (mA/cm²) | (mW/cm²) |
| 20 | 0.9243 | 2.94e−4 | 2.42e−4 |
| 25 | 0.9871 | 4.77e−4 | 4.24e−4 |
| 30 | 0.9600 | 6.12e−4 | 5.27e−4 |
| 35 | 0.9680 | 7.00e−4 | 6.02e−4 |
| 40 | 0.9702 | 8.37e−4 | 7.30e−4 |
| 50 | 0.9480 | 6.13e−4 | 5.20e−4 |
| 60 | 0.9430 | 5.57e−4 | 4.69e−4 |
| 70 | 0.5972 | 2.38e−4 | 1.19e−4 |
| 80 | 0.2796 | 9.46e−5 | 1.70e−5 |
| 90 | 0.1038 | 3.49e−5 | 1.32e−7 |

### Example 9

### Preparation of Alkyl Modified Alginate

Alginate membranes incorporated with quaternary ammonium bromides were formed by co-casting the quaternary ammonium bromide with 3 wt. % alginate suspension. The polymer used was either ultra low, low, or medium molecular weight alginate. The mixture-casting solutions were prepared by adding the quaternary ammonium bromides to the 3 wt. % suspension. All mixture-casting solutions were prepared so the concentration of quaternary ammonium bromides is in excess of the concentration of carboxylic acid sites in the alginate suspension. After optimization, it was determined that the most stable and reproducible membrane has a quaternary ammonium bromide concentration that is three times the concentration of the exchange sites.

One milliliter of the casting solution was placed in a weighing boat and allowed to dry. 7.0 mL of 18 MΩ water were added to the weighing boats and allowed to soak overnight. The water was removed and the films were rinsed thoroughly with 18 MΩ water and dried. Then, the films were resuspended in 1.0 mL of methanol. Ammonium bromide salts of tetrapropylammonium (T3A), tetrapentylammonium (T5A), tetrahexylammonium (T6A), tetraheptylammonium (T7A), trimethylicosylammonium (TMICA), trimethyloctyldecylammonium (TMODA), trimethylhexyldecylammonium (TMHDA), trimethyltetradecylammonium (TMTDA), trimethyloctylammonium (TMOA), trimethyldodecylammonium (TMDDA), trimethyldecylammonium (TMDA), trimethylhexylammonium (TMHA) tetrabutylammonium (TBA), triethylhexylammonium (TEHA) were used as alginate modifiers to see which yielded the best micellar structure. The micellar structure is important for effective immobilization of an enzyme.

To determine the pore characteristics, three drops of each polymer were then placed on a slide and left to dry. After completely drying, they were soaked in 1 mM Ru(bpy)⁺² in ethanol for at least 3 hours. After being rinsed off with ethanol, the polymers were left to dry before being imaged with a fluorescence microscope to see the micellar structure. An example of the structure is shown in FIG. 12.

In another experiment, ultralow molecular weight alginate and dodecylamine were placed in 25% ethanol and refluxed to produce a dodecyl-modified alginate by amidation of the carboxylic acid groups.

### Example 10

### Preparation of Alginate Electrodes

A solution of 3 wt. % of an alginate polymer modified with a hydrophobic ammonium cation described in Example 9 is suspended in t-amyl alcohol and a solution of enzyme (e.g., alcohol dehydrogenase, glucose dehydrogenase, bilirubin oxidase, glucose oxidase) is added. This solution is pipeted onto an electrode material. This electrode material is typically a carbon cloth, or other carbon material.

### Example 11

### Alginate Biofuel Cells

A biofuel cell having an anode enzyme immobilized in a hydrophobically modified alginate is prepared by mixture casting a hydrophobically modified alginate with a solution of enzyme and buffer and pipeting the mixture on a carbon cloth, thus, forming a bioanode similar to those described above in Example 10. A biocathode comprising a hydrophobically modified Nafion® membrane as described above and in U.S. patent application Ser. No. 10/931,147 (published as U.S. Patent Application Publication No. 2005/0095466) can be used to form a biofuel cell having a bioanode and a biocathode. Alternatively, a biofuel cell having a cathode enzyme immobilized in a hydrophobically modified alginate is prepared by mixture casting a hydrophobically modified alginate with a solution of enzyme and buffer and pipeting the mixture on a carbon cloth, thus, forming a biocathode. A bioanode comprising a hydrophobically modified Nafion® membrane as described above and in U.S. patent application Ser. No. 10/617,452 (published as U.S. Patent Application Publication No. 2004/0101741) can be used to form a biofuel cell having a bioanode and a biocathode. In another embodiment, a biofuel cell can be prepared that has a cathode enzyme immobilized in a hydrophobically modified alginate prepared as described above and a bioanode having an anode enzyme immobilized in a hydrophobically modified alginate prepared as described above.

### Example 12

### Microfluidic Biofuel Cell

Masters for the production of PDMS micromolding channels are made by coating a 4-in. silicon wafer with SU-8 10 negative photoresist using a spin coater (Brewer Science, Rolla, Mo.) operating with a spin program of 1000 rpm for 30 seconds for micromolding channel. For flow channels, a spin program of 1750 rpm for 30 seconds is used with SU-8 50 negative photoresist. The photoresist is prebaked at 90° C. for 5 minutes prior to UV exposure for 4 minutes with a near-UV flood source (Autoflood 1000, Optical Associates, Milpitas, Calif.) through a negative film containing the micromolding channel or flow channel design structures (Jostens, Topeka, Kans.). The transparency is made from a computer design drawn in Freehand (PC Version 8.0, Macromedia Inc., San Francisco, Calif.). The design is transferred to a transparency using an image setter with a resolution of 2400 dpi by a printing service (Jostens, Topeka, Kans.). Following this exposure, the wafer is postbaked at 90° C. for 5 minutes and developed in Nano SU-8 developer. The wafers containing the desired design are rinsed with acetone and isopropanol in order to remove any excess, unexposed photoresist that may have remained on the silicon wafer. The thickness of the photoresist is measured with a profilometer (Alpha Step-200, Tencor Instruments, Mountain View, Calif.), which corresponds to the channel depth of the PDMS structures.

A degassed 10:1 mixture of Sylgard 184 elastomer and curing agent are then poured onto the silicon wafer and cured at 75° C. for approximately 2 hrs. The PDMS is removed from the master wafer by cutting around the edges and peeling back the PDMS from the wafer. The master could be reused in order to generate numerous copies of the PDMS channels. The resulting PDMS flow channel is 200 mm wide, 100 mm deep and 3.0 cm long.

Soda-lime glass plates are purchased from a local glass shop. The plates were 7 cm wide, 10 cm long and 1.54 mm thick. The glass plates are cleaned by soaking them for 15 minutes in piranha solution (70% concentrated H₂SO₄/30% H₂O₂) to remove organic impurities. Glass is then rinsed thoroughly with Nanopure (18 MΩ-cm) water and dried with nitrogen. Using traditional lithographic and sputtering procedures, palladium electrodes are fabricated on the glass in specific patterns. Each plate could hold several flow channels with electrodes. This is more specifically accomplished by argon ion sputtering of a layer of titanium, for adhesive properties, and a layer of palladium. In order to accomplish this, the glass is placed into a deposition system (Thin Film Deposition System, Kurt J. Lesker Co.) for deposits of the metals. The thickness of the metals is monitored using a quartz crystal deposition monitor (Inficon XTM/2, Leybold Inficon). Titanium is deposited from a Ti-target at a rate of ˜2.3 angstroms/s to a depth of 200 angstroms. Palladium is deposited from a Pd-target at a rate of ˜1.9 angstroms/s to a depth of 2000 angstroms. AZ 1518 positive photoresist is dynamically dispensed onto the palladium coated glass. A pre-exposure bake at 95° C. for 1 minute is followed by a 9 second ultra-violet exposure through a positive film. The film is removed and the glass placed in a commercially available developer (AZ 300 MIF developer) for 45 seconds. After rinsing with water and drying with nitrogen, the glass is post baked for 1 minute at 95° C. Wet etching is employed using Aqua regia (8:7:1 H₂O:HCl:HNO₃) to remove the unwanted palladium and a titanium etchant to remove unwanted titanium from the glass. Once completed, the glass is rinsed with acetone and isopropanol to remove the remaining photoresist and dried with nitrogen.

A flow access hole is drilled through each glass plate, while immersed under water, with a 1-mm diamond drill bit and a Dremel rotary tool (Dremel). The syringe connector portion of a leur adapter is removed with the Dremel rotary tool and accompanying cutting disc. After polishing with a sanding disc, the leur adapter is affixed to the glass plate with J.B. Weld. The epoxy is cured in an oven (75° C.) for 2 hours before use. Connections are made to the palladium electrodes by copper wire and colloidal silver.

To fabricate carbon ink microelectrodes, first the PDMS micromolding channel is sealed to the glass plate in contact with the palladium leads (with leur fitting attached) that had been thoroughly cleaned. The PDMS channels are first primed with solvent thinner (N-160). The thinner is removed by applying a vacuum to one of the reservoirs. As soon as the thinner had been removed, a mixture of commercially available carbon ink and solvent thinner is added to the channels and pulled through the channel by applying vacuum (via water aspirator) to the opposite end. The ink/thinner mixture is made so that the volume of added thinner is 0.2% (v/w) of the initial ink weight. After filling channels with carbon ink, the reservoir where vacuum had been applied is filled with the ink/thinner solution and the entire chip placed in an oven at 75° C. for one hour. After this period of time, the PDMS could be removed from the glass, leaving the carbon microelectrode attached to the glass surface A final curing/conditioning step is achieved by placing the chip in a separate oven at 12° C. for one hour. The height of the carbon microelectrode is measured with a profilometer and the width is measured via microscopy.

In order to further characterize the carbon ink electrodes, cyclic voltammetry is employed and performed in a 3-electrode format using a CH Instruments 810 bipotentiostat (Austin, Tex.). The carbon microelectrode is the working electrode with a silver/silver chloride reference electrode and a platinum wire as the auxiliary electrode. A static cell for cyclic voltammetry experiments is created in a piece of PDMS by cutting a small section (1 cm×2 cm) out of a larger piece of PDMS (˜2 cm×3 cm); this piece of PDMS is then sealed over the carbon electrode so the entire length of the electrode is exposed to solution. For flow experiments, a PDMS microchannel (˜200 mm wide, 100 mm deep and ˜2 cm long) is sealed over the carbon electrode, so the entire electrode is sealed inside the microchannel. The auxiliary and reference electrodes are contained in the outlet reservoir by use of an electrochemical cell holder (CH Instruments).

The carbon working electrodes are electropolymerized with methylene green. Methylene green is a well-known electrocatalyst for NADH. The thin film of poly(methylene green) is formed by performing cyclic voltammetry using a CH Instruments Model 810 potentiostat (Austin, Tex.) from −0.3 V to 1.3 V for 7 scans cycles at a scan rate of 0.05 V/s in a solution containing 0.4 mM methylene green and 0.1 M sodium nitrate in 10 mM sodium borate. A piece of PDMS is used to define the electrochemical cell over the entire carbon electrode. A calomel reference electrode with a platinum wire auxiliary electrode completed the electrochemical cell. The electrode is rinsed and then allowed to dry overnight before further modification.

The flow access hole drilled in the glass plate allows for access to flow from a syringe pump (Pump 11, Harvard Apparatus, Holliston, Mass.). A syringe is filled with the solution of choice and placed in the syringe pump. With the use of high pressure fittings, leur adapters, and Teflon PEEK tubing, the syringe is connected to the glass microchip. The flow rates are varied from 0 μL/min to 15 μL/min through the 200 μm-wide PDMS flow channel which is aligned with one end at the flow access hole. The channel is sealed directly over the electrode. At the other end of the channel, a reservoir is formed by a hole punch and is where the cathode or reference and counter electrodes are placed.

The carbon ink electrode generally is a 2.5 cm long electrode that is 55 μm wide and 87 μm high. A solution of 1 mM tris(2,2′-bipyridyl)dichlororuthenium(II) hexahydrate and 0.1 M sodium sulfate as the electrolyte is used to characterize the response of the electrode using cyclic voltammetry. As flow rate is increased, the current density increased which is expected due to the analyte reaching the electrode surface faster with an increase in flow rates. Initially, an electrochemical pretreatment is utilized to clean the electrode by applying 1.5 V for 3 minutes in a 0.05 M phosphate buffer (pH 7.4).

Methylene green is immobilized onto the carbon microelectrodes using 14 scan segments from −0.3 V to 1.3 V, the same procedure employed for macro-scale carbon electrodes. Using commercially available microfittings, it is possible to pump flow rates up to 20 mL/min through 3 cm by 240 mm by 100 mm PDMS channels that are reversibly sealed over the carbon microelctrode. NADH is pumped through the PDMS flow channels at various flow rates of 0.5 mL/min to 15.0 mL/min.

The procedure above is followed with slight modification to simplify the process of forming an electrode comprising an electron conductor and an enzyme immobilization material. To do so, the electron conductor solution is modified to include the enzyme immobilization material. The additional material is prepared by adding a 2 wt. % solution of a hydrophobically modified chitosan in t-amyl alcohol or a 3 wt. % solution of a hydrophobically modified alginate in alcohol solution is suspended in Ercon N160 Solvent Thinner and vortexed thoroughly. Finally, 1 mL of this modified thinner is added to 0.5 g Ercon E-978(1) carbon-based ink. This modified electron conductor solution is then flowed through the mold cavity formed by the casting mold and the substrate and cured according to the method described above in this example.

To form a bioanode according to the invention, the general steps above in this example are used, with the anode being completed by flowing additional materials over the electron conductor after its curing and activation stages. To start, a solution of methylene green is made by syringe pumping across electron conductor. The solution is then electropolymerized for fourteen scan segments from −0.3 V to 1.3 V at a scan rate of 50 mV/s. Next, a casting solution of the remaining anode elements is created by combining a 2 wt. % solution of hydrophobically modified chitosan in t-amyl alcohol or 3 wt. % solution of hydrophobically modified alginate in alcohol, an enzyme solution, and an electron mediator in lower aliphatic alcohol. This solution is then vortexed together thoroughly and pumped through the approximately 100 mm microchannel at a flow rate of about 1 mL/min. The electron conductor and the casting solution are then allowed to dry overnight.

For the biocathode, the microchips and channel masters are fabricated as described above in this example using photolithography. The carbon ink microelectrodes generated from the micromolding procedure could be further modified with the hydrophobically modified chitosan membrane or hydrophobically modified alginate mixture described above.

The carbon microelectrodes are modified to serve as a bioanode. A hole is punched in PDMS to form a bulk reservoir that is placed around the microelectrode and include Ag/AgCl reference electrode and a platinum wire as the auxiliary electrode. Specifically, this is a static cell. A solution of 0.4 mM methylene green and 0.1 M sodium nitrate in 10 mM sodium borate is pipetted into the PDMS reservoir. Polymerization of methylene green via cyclic voltammetry is performed using a CH Instruments 650 potentiostat (Austin, Tex.) from −0.3V to 1.3V for 14 scan segments at a scan rate of 50 mV/s. The methylene green solution is pipetted out of the reservoir and the PDMS removed. The poly(methylene green) modified carbon ink microelectrodes are then rinsed with 18MΩ (Nanopure) water and allowed to dry overnight.

The enzyme/hydrophobically modified chitosan mixture or enzyme/hydrophobically modified alginate mixture is immobilized onto the carbon microelectrode using microchannels that are reversibly sealed over the microelectrodes and hydrodynamic flow. The size of this flow channel is such that alignment over the microelectrode is possible but is not much wider than the electrode. To accomplish this, a PDMS microchannel (130 mm wide, 100 mm deep and ˜2 cm long) is sealed over the carbon electrode (˜40 mm wide, ˜2 cm long, and ˜100 mm high), so that the entire electrode is sealed inside the microchannel. A 2:1 ratio of enzyme and hydrophobically modified chitosan mixture (or hydrophobically modified alginate mixture) with 1 mg of electron mediator for each 20 mL of hydrophobically modified chitosan (or hydrophobically modified alginate mixture) is prepared and vortexed until sufficiently mixed. The mixture is introduced to the channels thru a syringe by use of a syringe pump (Harvard Apparatus, Brookfield, Ohio) at 1.0 mL/min. Once the mixture travels the entire length of the channel (monitored visually), the solvent is allowed to evaporate at room temperature. This is possible since PDMS is permeable to gases. After evaporation is complete, the PDMS is removed, leaving a coated bioanode.

To form a biocathode according to the invention, the general steps described in this example were used, with the biocathode being completed by flowing additional materials over the electron conductor after its curing and activation stages.

To modify the electron conductor, a casting solution of bilirubin, bilirubin oxidase, and a hydrophobically modified chitosan (or hydrophobically modified alginate mixture) is vortexed together for about 20 minutes. Next, the solution is pumped through the approximately 100 mm microchannel at a flow rate of about 1 mL/min. The electron conductor and the casting solution are then allowed to dry overnight. Once dried, the electrode is soaked in a solution of Ru(bpy)₃⁺² and sodium sulfate for about 24 hours.

The biocathode is created in a similar fashion to the bioanode described above. A PDMS microchannel is sealed over a carbon ink microelectrode. Hydrophobically modified chitosan (or hydrophobically modified alginate) is mixed with an electron mediator and cathode enzyme. The mixture is then pumped through the channel at a 1.0 mL/min until it reached the end of the channel after which time the solvent is allowed to evaporate. Tris(2,2′-bipyridyl)dichlororuthenium(II) hexahydrate is exchanged within the membrane by flowing a 1.0 mM solution of it at a flow rate of 0.5 mL/min for 5 hours. Afterwards the PDMS flow channel is removed leaving a coated electrode that is used as a biocathode.

In view of the above, it will be seen that the several objects of the invention are achieved and other advantageous results attained.

As various changes could be made in the above methods without departing from the scope of the invention, it is intended that all matter contained in the above description or shown in the accompanying drawings shall be interpreted as illustrative and not in a limiting sense.

Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

1-71. (canceled)

72. A bioanode comprising
(a) an electron conductor;
(b) at least one anode enzyme capable of reacting with an oxidized form of an electron mediator and the fuel fluid to produce an oxidized form of the fuel fluid and a reduced form of the electron mediator, the reduced form of the electron mediator being capable of releasing electrons to the electron conductor; and
(c) an enzyme immobilization material being permeable to the fuel fluid and the electron mediator;
wherein said enzyme immobilization material comprises a hydrophobically modified polysaccharide.

73. The bioanode of claim 72 wherein the polysaccharide either:
(a) comprises chitosan, cellulose, chitin, starch, amylose, alginate, or a combination thereof,
(b) is capable of immobilizing and stabilizing the enzyme; or
(c) has a micellar structure.

74. The bioanode of claim 72 wherein the enzyme immobilization material comprises either
(a) a hydrophobically modified alginate wherein the hydrophobically modified alginate is modified with a hydrophobic cation larger than NH₄⁺; or
(b) a micellar hydrophobically modified polysaccharide corresponding to Formula 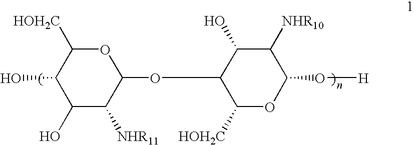
wherein n is an integer;
R₁₀ is independently hydrogen, hydrocarbyl, substituted hydrocarbyl, or a hydrophobic redox mediator; and
R₁₁ is independently hydrogen, hydrocarbyl, substituted hydrocarbyl, or a hydrophobic redox mediator.

75. The bioanode of claim 74 wherein R₁₀ is independently hydrogen or alkyl and R₁₁ is independently hydrogen or alkyl.

76. The bioanode of claim 72 wherein the enzyme immobilization material comprises the electron mediator.

77. A bioanode comprising
(a) an electron conductor;
(b) at least one anode enzyme capable of reacting with an oxidized form of an electron mediator and the fuel fluid to produce an oxidized form of the fuel fluid and a reduced form of the electron mediator;
(c) an enzyme immobilization material being permeable to the fuel fluid and the electron mediator; and
(d) an electrocatalyst adjacent the electron conductor, an oxidized form of the electrocatalyst being capable of reacting with the reduced form of the electron mediator to produce an oxidized form of the electron mediator and a reduced form of the electrocatalyst, the reduced form of the electrocatalyst being capable of releasing electrons to the electron conductor;
wherein said enzyme immobilization material comprises a hydrophobically modified polysaccharide.

78. A biocathode comprising:
(a) an electron conductor;
(b) at least one cathode enzyme capable of reacting with a reduced form of an electron mediator and an oxidant to produce an oxidized form of the electron mediator and water; and
(c) an enzyme immobilization material comprising an electrocatalyst, an electron mediator, or an electrocatalyst and an electron mediator, the enzyme immobilization material being permeable to the oxidant, an oxidized form of the electrocatalyst being capable of gaining electrons from the electron conductor to produce a reduced form of the electrocatalyst that is capable of reacting with an oxidized form of the electron mediator to produce a reduced form of the electron mediator and an oxidized form of the electrocatalyst;
wherein said enzyme immobilization material comprises a hydrophobically modified polysaccharide.

79. The biocathode of claim 78 wherein the polysaccharide either:
(a) comprises chitosan, cellulose, chitin, starch, amylose, alginate, or a combination thereof,
(b) is capable of immobilizing and stabilizing the enzyme; or
(c) has a micellar structure.

80. The biocathode of claim 78 wherein the enzyme immobilization material comprises either
(a) a hydrophobically modified alginate wherein the hydrophobically modified alginate is modified with a hydrophobic cation larger than NH₄⁺; or
(b) a micellar hydrophobically modified polysaccharide corresponding to Formula 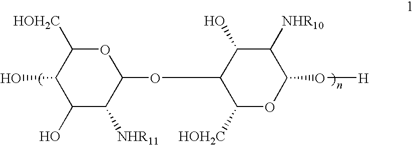
wherein n is an integer;
R₁₀ is independently hydrogen, hydrocarbyl, substituted hydrocarbyl, or a hydrophobic redox mediator; and
R₁₁ is independently hydrogen, hydrocarbyl, substituted hydrocarbyl, or a hydrophobic redox mediator.

81. The biocathode of claim 80 wherein R₁₀ is independently hydrogen or alkyl and R₁₁ is independently hydrogen or alkyl.

82. A biocathode comprising:
(a) an electron conductor;
(b) at least one cathode enzyme capable of reacting with a reduced form of an electron mediator and an oxidant to produce an oxidized form of the electron mediator and water; and
(c) an enzyme immobilization material comprising the electron mediator, the enzyme immobilization material being permeable to the oxidant, an oxidized form of the electron mediator being capable of gaining electrons from the electron conductor to produce a reduced form of the electron mediator;
wherein said enzyme immobilization material comprises a hydrophobically modified polysaccharide.

83. A biofuel cell for generating electricity comprising:
a fuel fluid;
an electron mediator;
a bioanode of claim 72; and
a cathode.

84. A biofuel cell for generating electricity comprising:
a fuel fluid;
an electron mediator;
an anode; and
a biocathode of claim 78.

85. A biofuel cell of claim 83 wherein the cathode comprises a biocathode of claim 78.

86. An enzyme immobilized in a micellar hydrophobically modified polysaccharide, the micellar hydrophobically modified polysaccharide being capable of immobilizing and stabilizing the enzyme, the micellar hydrophobically modified polysaccharide being permeable to a compound smaller than the enzyme.

87. The immobilized enzyme of claim 86 wherein the polysaccharide comprises chitosan or alginate.

88. The immobilized enzyme of claim 86 wherein the micellar hydrophobically modified polysaccharide corresponds to Formula 1 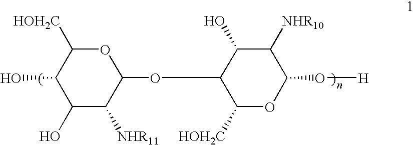
wherein n is an integer;
R₁₀ is independently hydrogen, hydrocarbyl, substituted hydrocarbyl, or a hydrophobic redox mediator; and
R₁₁ is independently hydrogen, hydrocarbyl, substituted hydrocarbyl, or a hydrophobic redox mediator.

89. The immobilized enzyme of claim 88 wherein R₁₀ is independently hydrogen or alkyl and R₁₁ is independently hydrogen or alkyl.

90. The immobilized enzyme of claim 88 wherein R₁₀ is independently hydrogen or hexyl and R₁₁ is independently hydrogen or hexyl.

91. A micellar hydrophobic redox mediator modified chitosan having a structure corresponding to Formula 1A 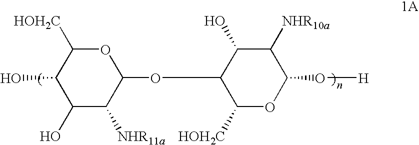
wherein n is an integer;
R₁₀ₐ is independently hydrogen, or a hydrophobic redox mediator; and
R₁₁ₐ is independently hydrogen or a hydrophobic redox mediator.

92. The modified chitosan of claim 91 wherein the hydrophobic redox mediator is Ru(phen)₃⁺², Fe(phen)₃⁺², Os(phen)₃⁺², Co(phen)₃⁺², Cr(phen)₃⁺², Ru(bpy)₃⁺², Os(bpy)₃⁺², Fe(bpy)₃⁺², Co(bpy)₃⁺², Cr(bpy)₃⁺², Os(terpy)₃⁺², Ru(bpy)₂(4-methyl-4′-(6-hexyl)-2,2′-bipyridine)⁺², Co(bpy)₂(4-methyl-4′-(6-hexyl)-2,2′-bipyridine)⁺², Cr(bpy)₂(4-methyl-4′-(6-hexyl)-2,2′-bipyridine)⁺², Fe(bpy)₂(4-methyl-4′-(6-hexyl)-2,2′-bipyridine)⁺², Os(bpy)₂(4-methyl-4′-(6-hexyl)-2,2′-bipyridine)⁺², or a combination thereof.
